(19) United States
(12) Patent Application Publication
(10) Pub. No.: US 2023/0120988 A1
(43) Pub. Date: Apr. 20, 2023

(54) CORONA NUCLEOCAPSID ANTIGEN FOR USE IN ANTIBODY-IMMUNOASSAYS

(71) Applicant: Roche Diagnostics Operations, Inc., Indianapolis, IN (US)

(72) Inventors: Frederik Baumkoetter, Bavaria (DE); Juliane Benz, Weilheim (DE); Markus Eckl, Penzberg (DE); Peter Muench, Penzberg (DE); Alexander Riedel, Penzberg (DE); Christian Scholz, Penzberg (DE)

(21) Appl. No.: 17/907,039

(22) PCT Filed: Apr. 22, 2021
(86) PCT No.: PCT/EP2021/060578

§ 371 Date: Sep. 22, 2022

Related U.S. Application Data
(63) Continuation of application No. 16/867,750, filed on May 6, 2020, now abandoned.
Application No. 16/867,750 is a continuation of application No. 16/856,162, filed on Apr. 23, 2020, now abandoned.

(30) Foreign Application Priority Data
Apr. 23, 2020 (EP) 20171154.6
May 6, 2020 (EP) 20173315.1
Jun. 8, 2020 (EP) 20178739.7

(51) Int. Cl. G01N 33/569; C07K 14/005

(52) U.S. Cl. CPC G01N 33/56983; C07K 14/005; G01N 2800/26; C12N 2770/20052; G01N 2333/165; C12N 2770/20022; G01N 2469/20

(57) ABSTRACT

The present invention relates to a Corona antigen comprising a Corona nucleocapsid specific amino acid sequence, compositions, and reagent kits comprising the same and methods of producing it. Also encompassed are methods of detecting anti-Corona antibodies in samples using said Corona antigen, and methods of differential diagnosis of an immune response in a patient due to natural Corona infection or due to vaccination against Corona.

### CROSS-REFERENCE TO RELATED APPLICATIONS

This application claims priority to International Application No. PCT/EP2021/060578 filed Apr. 22, 2021, which claims priority to European Application 20178739.7 filed Jun. 8, 2020, U.S. patent application Ser. No. 16/867,750 filed May 6, 2020, European Application 20173315.1 filed May 6, 2020, U.S. patent application Ser. No. 16/856,162 filed Apr. 23, 2020, and European Application 20171154.6 filed Apr. 23, 2020, the disclosures of which are hereby incorporated by reference in their entirety.

The present invention relates to a Corona antigen comprising a Corona nucleocapsid specific amino acid sequence, compositions, and reagent kits comprising the same and methods of producing it. Also encompassed are methods of detecting antiCorona antibodies in samples using said Corona antigen, and methods of differential diagnosis of an immune response in a patient due to natural Corona infection or due to vaccination against Corona.

### BACKGROUND

The SARS Corona-2 virus was discovered by Chinese virologists in the end of 2019 and has, since then, spread relentlessly throughout the world. Formerly known as nCoV-19 (novel Corona virus 2019), SARS CoV-2, the etiological agent of the Coronavirus Disease 2019 (COVID-19) triggered a pandemic in early 2020 leading to substantial restrictions of public life and severe economic effects worldwide.

Diagnostic tests allowing for the detection of acutely infected patients were made available rapidly. However, the quantities of tests available could by no means satisfy the high demand during the pandemic. Thus, many patients outside of clinics and hospitals were not tested as available tests were primarily reserved for those patients with highly critical conditions. Statistically, 4 of 5 patients infected with SARS CoV-2 develop only mild symptoms such as a mild sore throat, dry cough, or mild fever. As a consequence, it is currently unknown how many people were or still are infected and how many have already recovered from the infection.

To evaluate the extent of the current pandemic, it would be very helpful to be able to assess the infection rate and hence the true mortality rate of SARS-2 correctly. Furthermore, patients known to have recovered from the disease and to have acquired immunity could be excluded from public lockdowns and help those still in need, e.g. in clinics and hospitals.

Immunological tests able to detect antibodies against SARS CoV-2 virus in patients are thus desperately needed. Such antibody tests allow for the identification of patients who were affected with the infection previously, potentially with such mild progression of the disease that they were not even aware of it. Accordingly, such tests would allow to evaluate, reliably and for the first time, the true infection rate both within different cohorts and within the population as a whole. Further, such tests would allow to assess whether vaccines developed against SARS CoV-2 virus infection are actually effective in stimulating an immune response in patients, and are therefore utterly needed in the assessment of the success of vaccination campaigns.

Yet, automated high-throughput assays to detect anti-SARS CoV-2 antibodies in patients with the required sensitivity and specificity are still not available. With the currently approved antibody tests, no more than one third of the infected patients could be diagnosed correctly, whilst two thirds of the infected patients received false reports. One of the main issues here is to equip the test with antigens which are able to be recognized by anti-SARS CoV-2 antibodies with both high sensitivity and specificity.

Several Corona antigens are known in the art since the first reported appearance of SARS in 2002/2003. The Spike protein of Corona, and in particular its receptor binding domain (RBD), is considered to be the most promising candidate as it was shown previously to be highly immunologically reactive (Wang et al.(Clin Chem (2003) 49 (12), 1989-1996); and He et al. (J. Clin. Microbiol. (2004) 42 (11), 5309-5314), i.e., a strong antibody response is mounted against the RBD in the course of the humoral immune response upon infection with SARS CoV. As a consequence, the receptor binding domain also serves as the main antigen in current assay developments (Amanat et al., medRxiv, Mar. 18, 2020). In this very recent manuscript, the authors describe the use of the CoV-2 receptor binding domain as a capture antigen in an ELISA format. However, the sensitivity data have been determined based on only four positive sera (from three COVID-19 patients), and the specificity data rely on 59 negative sera only. The amount of samples analysed is too little to allow for statements regarding sensitivity and specificity with statistical significance. Furthermore, antibody assays in an ELISA format often require time-consuming and laborious manual steps and often high throughput applications are precluded by the limited availability of the assay.

Contrary to the prejudice in the prior art that antigens derived from the spike protein are most promising for the development of a Corona antibody assay, the current invention relates to an immunological test using the nucleocapsid protein of the SARS CoV-2 virus as an antigen for the reliable detection of anti-SARS-CoV-2 antibodies. Surprisingly, the inventors could show that by using the nucleocapsid protein of SARS CoV-2 as an antigen, both a high sensitivity and a high specificity of the resulting immunological test could be achieved allowing for the development of the urgently needed and eagerly awaited automated high-throughput Corona antibody assay.

### SUMMARY OF THE INVENTION

In a first aspect, the present invention relates to a Corona antigen suitable for detecting antibodies against Corona virus in an isolated biological sample comprising a Corona nucleocapsid specific amino acid sequence, in particular a Corona nucleocapsid specific amino acid sequence according to SEQ ID NO: 1 or a Corona nucleocapsid specific amino acid sequence having 95% sequence homology to the amino acid sequence of SEQ ID NO: 1. In particular said polypeptide comprises no further Corona virus specific amino acid sequences.

In a second aspect, the present invention relates to a composition comprising the Corona antigen of the first aspect of the present invention In a third aspect, the present invention relates to a method of producing a Corona antigen specific for Corona virus nucleocapsid, said method comprising the steps of a) culturing host cells, in particular E. coli cells, transformed with an expression vector comprising operably linked a recombinant DNA molecule encoding the antigen of the first aspect of the present invention, in particular a recombinant DNA molecule comprising a sequence according to SEQ ID NO: 3
b) expression of said polypeptide and
c) purification of said polypeptide.

In a fourth aspect, the present invention relates to a method for detecting antibodies specific for Corona virus in an isolated sample, wherein a Corona antigen of the first aspect of the present invention, the composition of the second aspect of the present invention, or a Corona antigen obtained by a method of the third aspect of the present invention is used as a capture reagent and/or as a binding partner for said anti-Corona virus antibodies.

In a fifth aspect, the present invention relates to a method for detecting antibodies specific for Corona virus in an isolated sample said method comprising
a) forming an immunoreaction mixture by admixing a body fluid sample with a Corona virus antigen of the first aspect of the present invention, the composition of the second aspect of the present invention, or a Corona virus antigen obtained by the method of the third aspect of the present invention
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or the concentration of any of said immunoreaction product.

In a sixth aspect, the present invention relates to a method of identifying if a patient has been exposed to Corona virus infection in the past, comprising
a) forming an immunoreaction mixture by admixing a body fluid sample of the patient with a Corona virus antigen of the first aspect of the present invention, a composition of the second aspect of the present invention, or a Corona virus antigen obtained by the method of the third aspect of the present invention
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or absence of any of said immunoreaction product,

wherein the presence of an immunoreaction product indicates that the patient has been exposed to Corona virus infection in the past.

In a seventh aspect, the present invention relates to a method of differential diagnosis between an immune response in a patient due to natural Corona virus infection and an immune response due to vaccination, wherein the vaccination is based on S—, E—, or M-protein derived antigens, comprising
a) forming an immunoreaction mixture by admixing a body fluid sample of the patient with a Corona virus antigen of the first aspect of the present invention, a composition comprising the Corona Antigen of the first of the present invention, or a Corona virus antigen obtained by the method of the third aspect of the present invention
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or absence of any of said immunoreaction product,

wherein the presence of an immunoreaction product indicates that the immunresponse in the patient is due to a natural Corona virus infection, and wherein the absence of a immunoreaction product indicates that the immunresponse in the patient is due to vaccination with spike protein derived antigens.

In an eighth aspect, the present invention relates to a use of a Corona antigen of the first aspect of the present invention, the composition of the second aspect of the present invention, or of a Corona antigen obtained by the method of the third aspect of the present invention in a high throughput in vitro diagnostic test for the detection of anti-Corona virus antibodies.

In a ninth aspect, the present invention relates to a reagent kit for the detection of anti-Corona virus antibodies, comprising a Corona antigen of the first aspect of the present invention, the composition of the second aspect of the present invention, or a Corona antigen obtained by the method of the third aspect of the present invention.

### LIST OF FIG

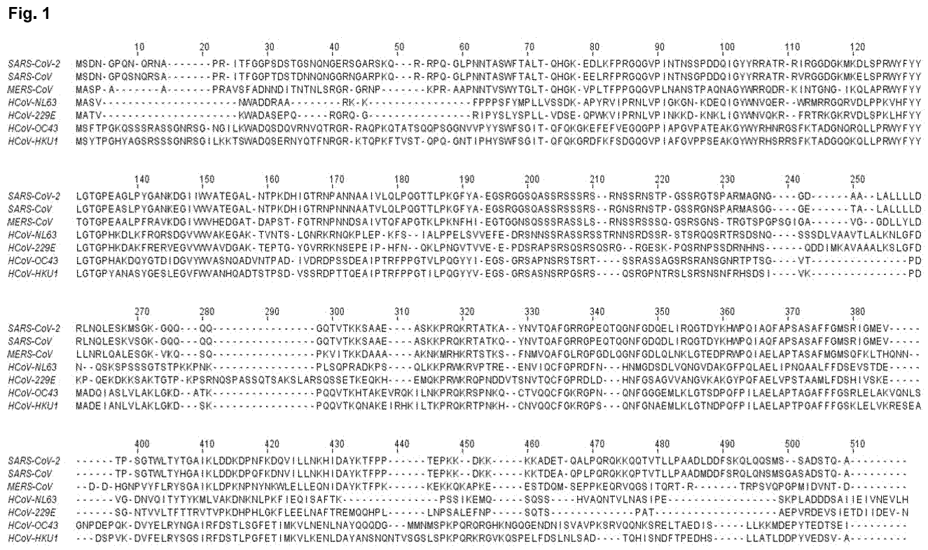

FIG. 1: Alignment of known Corona Virus Nucleocapsid sequences according to the following UniProt ID NOs, Gene Bank Acc NOs, and respective SEQ ID NOs:
Severe acute respiratory syndrome Coronavirus 2 N (SARS-CoV-2), (3-CoV: UniProt ID PODTC9; Gene Bank Acc.: MN908947; SEQ ID NO: 16
Severe acute respiratory syndrome Coronavirus N (SARS-CoV), β-CoV: UniProt ID P59595; Gene Bank Acc.: AY278741; SEQ ID NO: 17
Middle East respiratory syndrome-related Coronavirus N (MERS-CoV), (3-CoV: UniProt ID T2BBK0; Gene Bank Acc.: KF600632; SEQ ID NO: 18
Human Coronavirus NL63 N (HCoV-NL63), α-CoV: UniProt ID Q6Q1R8; Gene Bank Acc: AY567487; SEQ ID NO: 19
Human Coronavirus 229E N (HCoV-229E), α-CoV: UniProt ID P15130; Gene Bank Acc: X51325; SEQ ID NO: 20
Human Coronavirus OC43 N (HCoV-OC43), β-CoV: UniProt ID P33469; Gene Bank Acc.: AY585228; SEQ ID NO: 21
Human Coronavirus HKU1 N (HCoV-HKU1), β-CoV: UniProt ID Q5MQC6; Gene Bank Acc.: AY597011; SEQ ID NO: 22

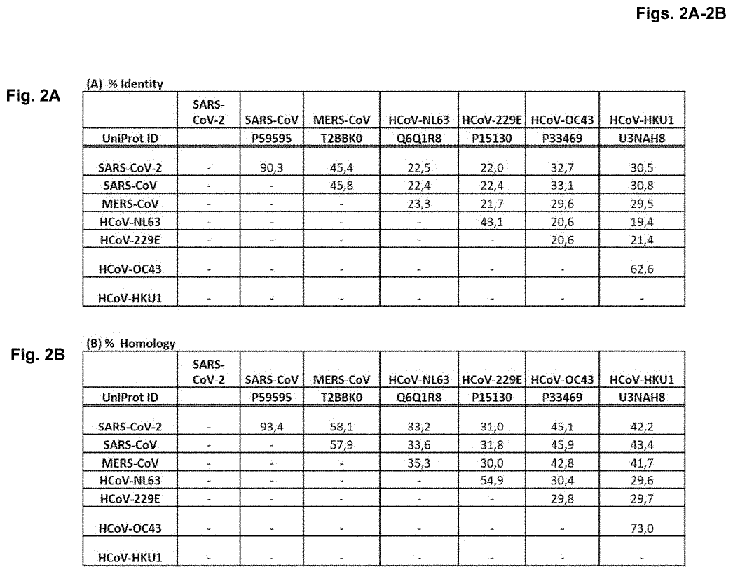

FIGS. 2A-2B: Sequence Comparison (FIG. 2A) Degree of sequence Identity (%) of SARS CoV-2 Nucleocapsid amino acid sequence to Nucleocapsid Sequence of different Corona Viruses; (FIG. 2B) Degree of sequence Homology (%) of SARS CoV-2 Nucleocapsid amino acid Sequence to Nucleocapsid Sequence of different Corona Viruses.

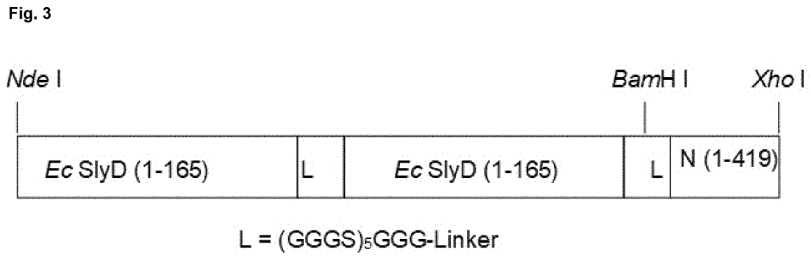

FIG. 3: Graphical representation of EcSlyD-EcSlyD-CoV-2 N (1-419) antigen

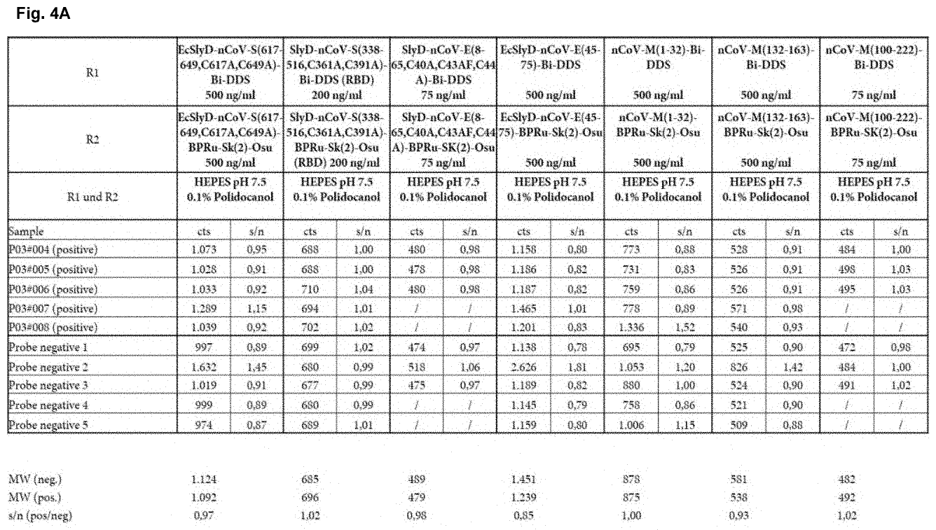

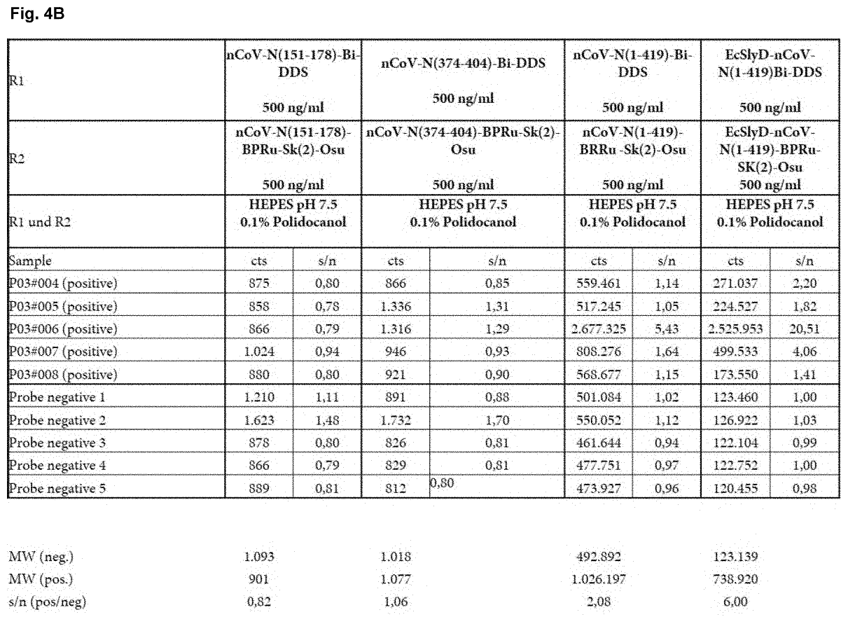

FIG. 4A: Comparison of immunological reactivity of antigens derived from Corona SARS CoV-2 S-, E—, and M-protein

FIG. 4B: Comparison of different antigens derived from SARS CoV-2 nucleocapsid protein

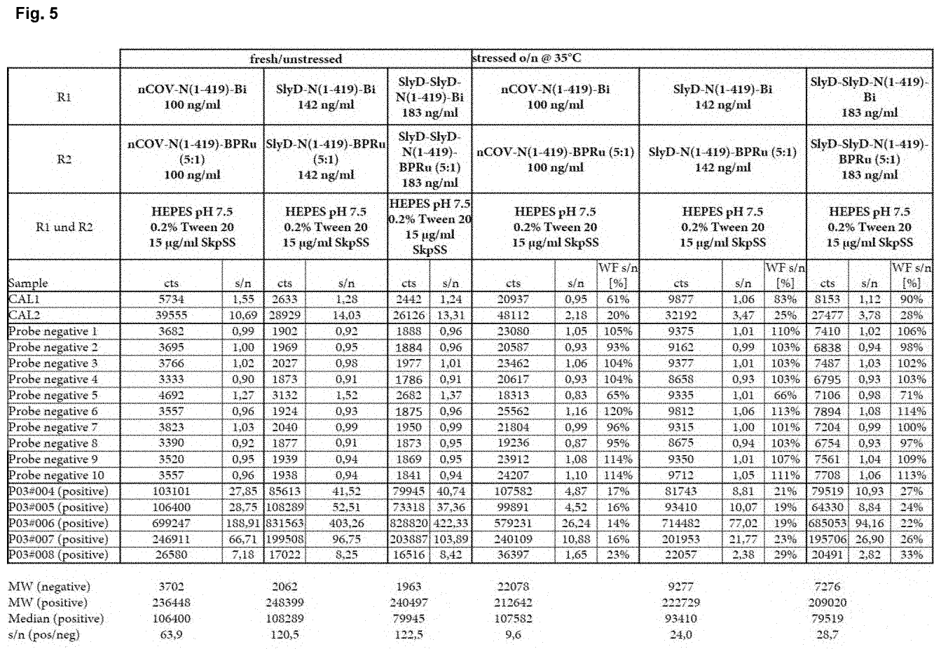

FIG. 5: Comparison of the immunological reactivity of full-length nucleocapsid fused to no, one, or two SlyD-chaperones

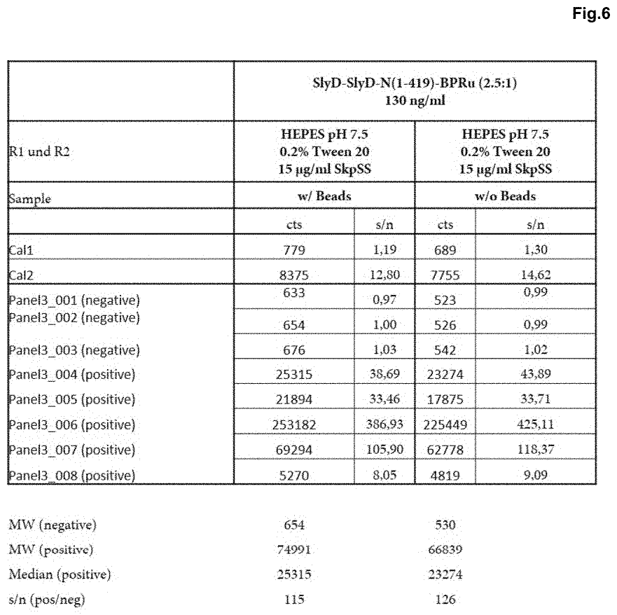

FIG. 6: Influence of bead pretreatment of the ruthenium conjugate (as an additional workflow in the production process) on assay performance

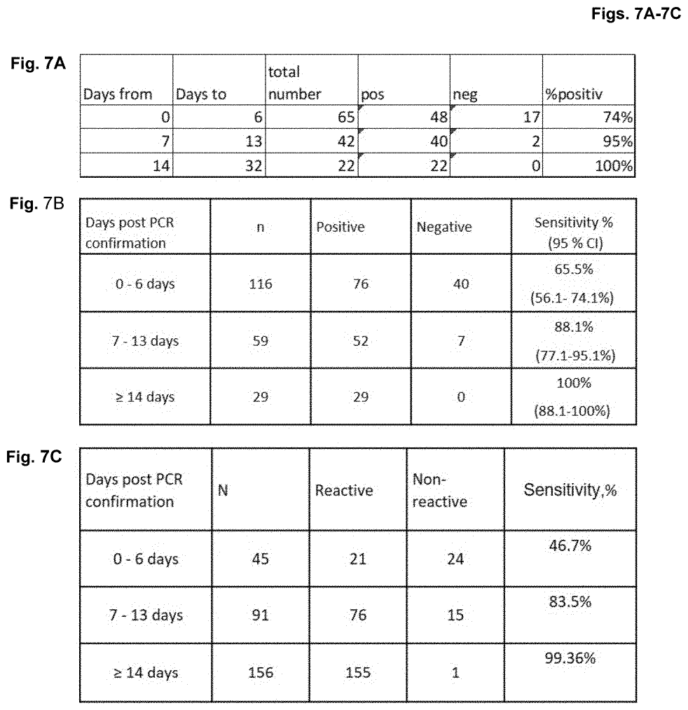

FIGS. 7A-7C: Sensitivity of the SARS CoV-2 assay; FIG. 7A) Initial results obtained from samples of 129 confirmed SARS CoV-2 patients; and FIG. 7B) further results including a total of 214 confirmed SARS CoV-2 patients; FIG. 7C) additional results of further 292 confirmed SARS CoV-2 patients

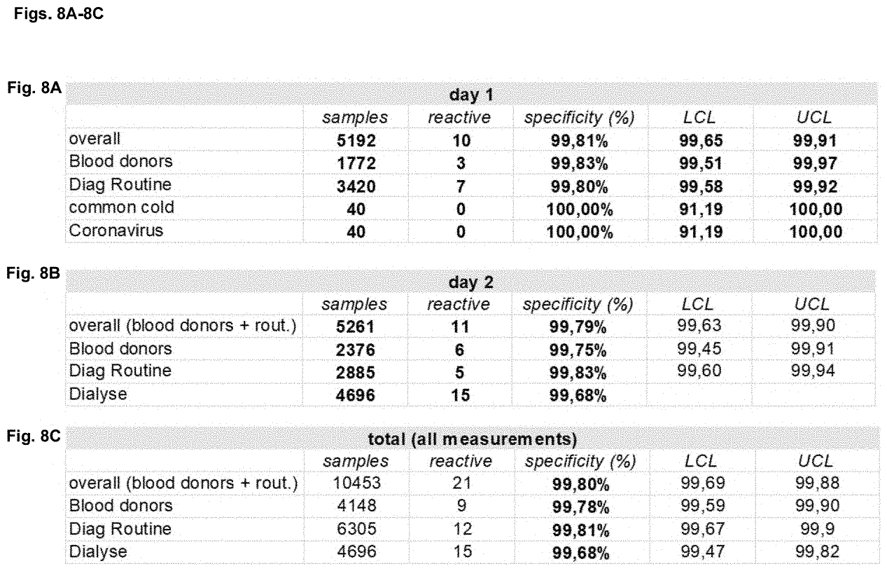

FIGS. 8A-8C: Specificity of the SARS CoV-2 assay; FIG. 8A) results of a first set of measured samples from 5192 patients and 80 potential cross reactive samples; FIG. 8B) results of a second set of measured samples from 5261 patients; and FIG. 8C) results from all patients (10453 in total). Common cold and Coronavirus cross reactive samples are not routine diagnostic or blood donors therefore these are excluded from total specificity calculation.

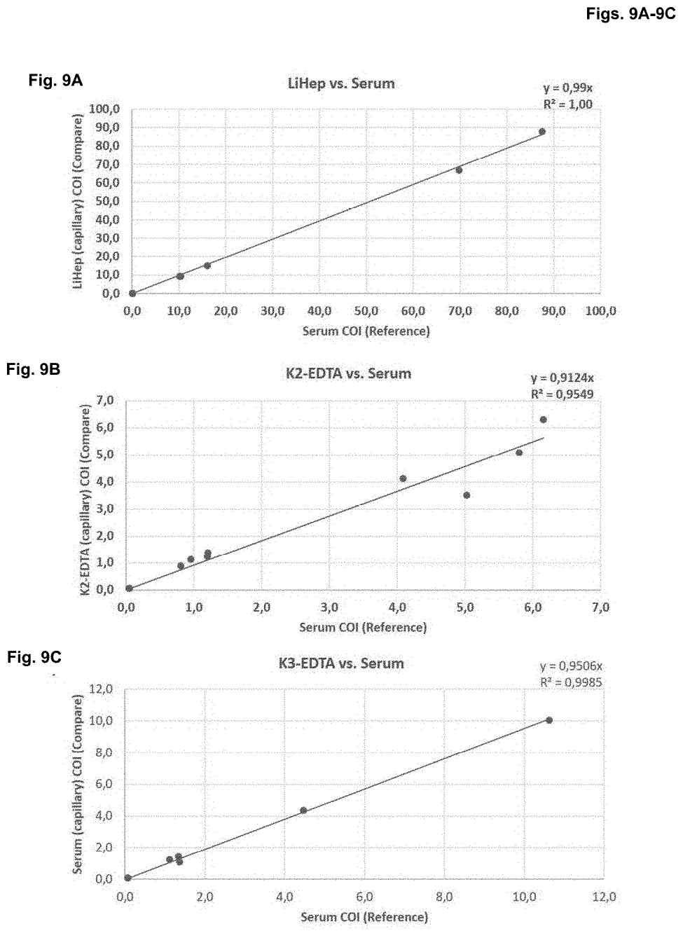

FIGS. 9A-9C: Correlation of the assay performance obtained with venous serum sample vs. capillary blood sample

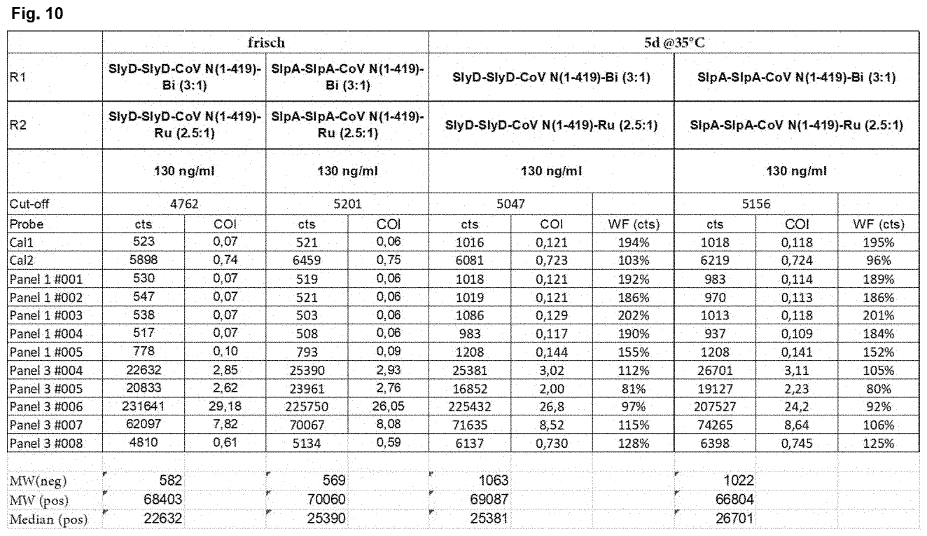

FIG. 10: Comparison of immune reactivity of antigens comprising sequence of SARS CoV-2nucleocapsid fused to two SlyD—, or to two SlpA-Chaperones

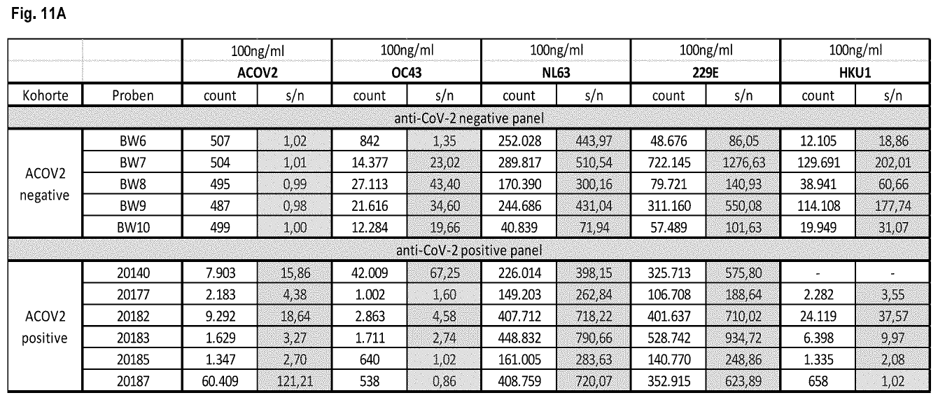

FIGS. 11A-11B: Reactivity of the N-terminal domains of the nucleocapsid protein from SARS-CoV-2, OC43, NL63, 229E and HKU1. The measurements were carried out in a DAGS format on a cobas e411 automated analyzer. The concentration of the biotin-conjugates (R1) and ruthenium conjugates (R2) were 100 ng/ml each. The signal readout (in counts) was normalized to the mean of the respective negative values to yield the signal dynamics (s/n).

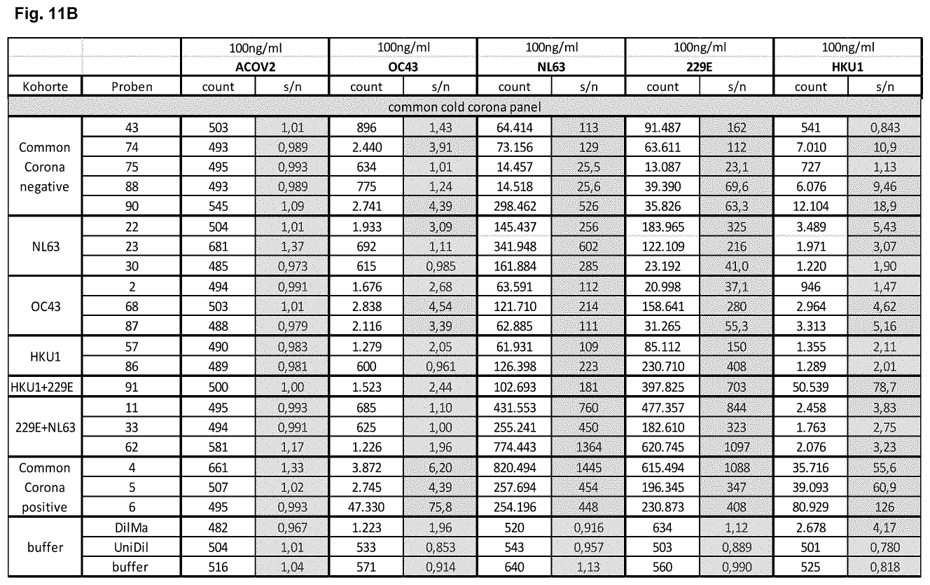

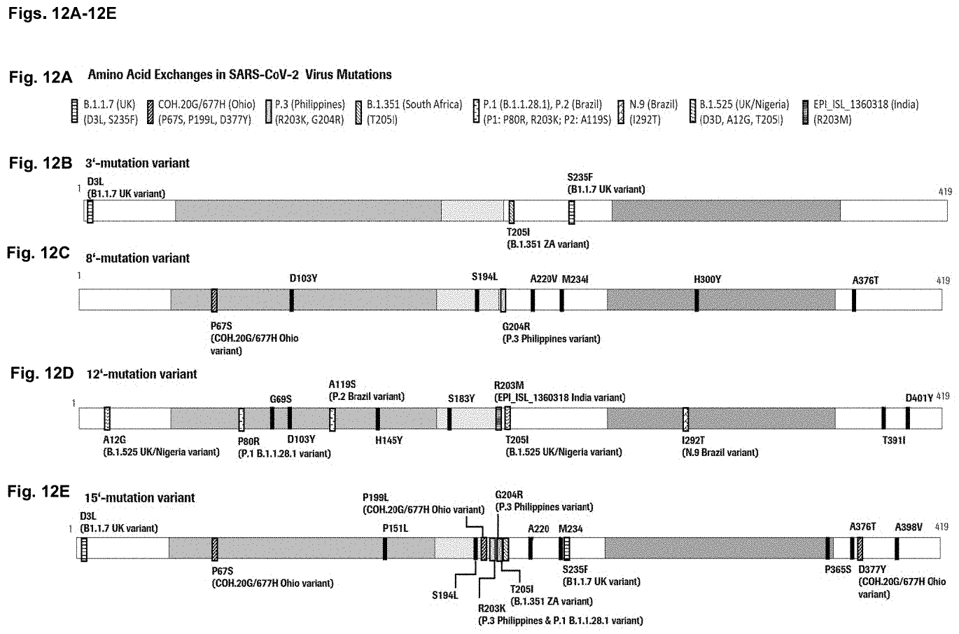

FIGS. 12A-12E: Schematic Drawing of 4 single-point mutation-variants of SARS CoV-2 nucleocapsid antigen

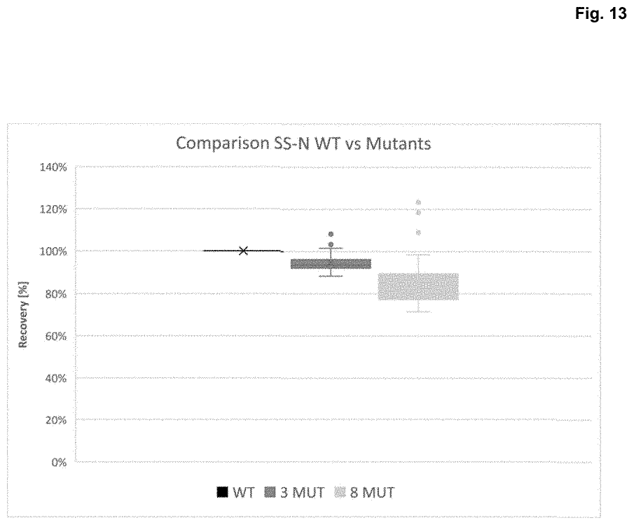

FIG. 13: Signal Recovery of WT vs. 3 MUT or 8 MUT single-point mutation-variants of SARS CoV-2 nucleocapsid antigen

### LIST OF SEQUENCES

SEQ ID NO: 1: Amino Acid Sequence of the Coronavirus SARS CoV-2 nucleocapsid
SEQ ID NO: 2: Amino Acid Sequence of the Coronavirus SARS CoV-2 nucleocapsid fused to one SlyD chaperone
SEQ ID NO: 3: Amino Acid Sequence of the Coronavirus SARS CoV-2 nucleocapsid fused to two SlyD chaperones
SEQ ID NO: 4: Nucleotide sequence of the Coronavirus SARS CoV-2 nucleocapsid
SEQ ID NO: 5: Nucleotide Sequence of the Coronavirus SARS CoV-2 nucleocapsid fused to one SlyD chaperone
SEQ ID NO: 6: Nucleotide Sequence of the Coronavirus SARS CoV-2 nucleocapsid fused to two SlyD chaperones
SEQ ID NO: 7: Linker Peptide
SEQ ID NO: 8: Amino Acid Sequence of the SARS CoV-2-N 3 MUT variant
SEQ ID NO: 9: Amino Acid Sequence of the EcSlyD-EcSlyD-SARS CoV-2-N 3 MUT variant
SEQ ID NO: 10: Amino Acid Sequence of the SARS CoV-2-N 8 MUT variant
SEQ ID NO: 11: Amino Acid Sequence of the EcSlyD-EcSlyD-SARS CoV-2-N 8 MUT variant
SEQ ID NO: 12: Amino Acid Sequence of the SARS CoV-2-N 12 MUT variant
SEQ ID NO: 13: Amino Acid Sequence of the EcSlyD-EcSlyD-SARS CoV-2-N 12 MUT variant
SEQ ID NO: 14: Amino Acid Sequence of the SARS CoV-2-N 15 MUT variant
SEQ ID NO: 15: Amino Acid Sequence of the EcSlyD-EcSlyD-SARS CoV-2-N 15 MUT variant
SEQ ID NO: 16: Amino Acid Sequence of Severe acute respiratory syndrome Coronavirus 2 (SARS CoV-2), β-CoV: UniProt ID PODTC9; Gene Bank Acc.: MN908947
SEQ ID NO: 17: Amino Acid Sequence of Severe acute respiratory syndrome Coronavirus (SARS CoV), β-CoV: UniProt ID P59595; Gene Bank Acc.: AY278741
SEQ ID NO: 18: Amino Acid Sequence of Middle East respiratory syndrome-related Coronavirus (MERS-CoV), β-CoV: UniProt ID T2BBK0; Gene Bank Acc.: KF600632
SEQ ID NO: 19: Amino Acid Sequence of Human Coronavirus NL63 (HCoV-NL63), α-CoV: UniProt ID Q6Q1R8; Gene Bank Acc: AY567487
SEQ ID NO: 20: Amino Acid Sequence of Human Coronavirus 229E (HCoV-229E), α-CoV: UniProt ID P15130; Gene Bank Acc: X51325
SEQ ID NO: 21: Amino Acid Sequence of Human Coronavirus OC43 (HCoV-OC43), β-CoV: UniProt ID P33469; Gene Bank Acc.: AY585228
SEQ ID NO: 22: Amino Acid Sequence of Human Coronavirus HKU1 (HCoV-HKU1), β-CoV: UniProt ID Q5MQC6; Gene Bank Acc.: AY597011

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an” and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include, but are not limited to an unpleasant sensation that may be felt as a persistent or varying burning, throbbing, itching or stinging ache.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. Examples of a disease include but are not limited to infectious diseases, inflammatory diseases, cutaneous conditions, endocrine diseases, intestinal diseases, neurological disorders, joint diseases, genetic disorders, autoimmune diseases, traumatic diseases, and various types of cancer.

The term “Coronaviruses” refers to a group of related viruses that cause diseases in mammals and birds. In humans, Coronaviruses cause respiratory tract infections that can range from mild to lethal. Mild illnesses include some cases of the common cold, while more lethal varieties can cause “SARS”, “MERS”, and “COVID-19”. Coronaviruses contain a positive-sense, single-stranded RNA genome.

The viral envelope is formed by a lipid bilayer wherein the membrane (M), envelope (E) and spike (S) structural proteins are anchored. Inside the envelope multiple copies of the nucleocapsid (N) protein form the nucleocapsid, which is bound to the positive-sense single-stranded RNA genome in a continuous beads-on-a-string type conformation. Its genome comprises Orfs 1a and 1b encoding the replicase/transcriptase polyprotein, followed by sequences encoding the spike (S)-envelope protein, the envelope (E)-protein, the membrane (M)-protein and the nucleocapsid (N)-protein. Interspersed between these reading frames are the reading frames for the accessory proteins which differ between the different virus strains.

Several human Coronaviruses are known, four of which lead to rather mild symptoms in patients:

Human Coronavirus NL63 (HCoV-NL63), α-CoV

Human Coronavirus 229E (HCoV-229E), α-CoV

Human Coronavirus HKU1 (HCoV-HKU1), β-CoV

Human Coronavirus OC43 (HCoV-OC43), β-CoV

Three human Coronaviruses produce symptoms that are potentially severe:

Middle East respiratory syndrome-related Coronavirus (MERS-CoV), β-CoV

Severe acute respiratory syndrome Coronavirus (SARS-CoV), β-CoV

Severe acute respiratory syndrome Coronavirus 2 (SARS-CoV-2), β-CoV

SARS CoV-2 causes Coronavirus disease 2019 (COVID-19). Because the strain was first discovered in Wuhan, China, it is sometimes referred to as the Wuhan virus.

SARS CoV-2 is highly contagious in humans, and the World Health Organization (WHO) has designated the still ongoing pandemic of COVID-19 a Public Health Emergency of International Concern. The earliest case of infection currently known is thought to have been found on 17 Nov. 2019. The SARS CoV-2 sequence was first published on Jan. 10, 2020 (Wuhan-Hu-1, GenBank accession number MN908947). Subsequent to the first outbreak in Wuhan, the virus spread to all provinces of China and to more than 150 other countries in Asia, Europe, North America, South America, Africa, and Oceania. Symptoms include high-fever, sore throat, dry cough, and exhaustion. In severe cases, pneumonia may develop.

The term “natural Corona virus” refers to a corona virus as occurring in nature, i.e. to any coronavirus as disclosed above. It is understood that a natural Corona virus comprises all proteins and nucleic acid molecules present in a naturally occurring virus. In difference to a natural Corona virus, “viral fragments”, “virus-like particles”, or Corona specific antigens, only comprise some but not all proteins and nucleic acid molecules present in a naturally occurring virus. Accordingly, such “viral fragments”, “virus-like particles”, or Corona specific antigens are not infectious but are still able to inflict an immune response in a patient. Accordingly, vaccination with Corona specific viral fragments, Corona specific virus-like particles, or Corona specific antigens inflicts the productions of antibodies against those viral fragments, virus-like particles, or antigens, in the patient.

As used herein, a “patient” means any mammal, fish, reptile or bird that may benefit from the diagnosis, prognosis or treatment described herein. In particular, a “patient” is selected from the group consisting of laboratory animals (e.g. mouse, rat, rabbit, or zebrafish), domestic animals (including e.g. guinea pig, rabbit, horse, donkey, cow, sheep, goat, pig, chicken, camel, cat, dog, turtle, tortoise, snake, lizard or goldfish), or primates including chimpanzees, bonobos, gorillas and human beings. It is particularly preferred that the “patient” is a human being.

The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, urine, saliva, and lymphatic fluid, or solid samples such as tissue extracts, cartilage, bone, synovium, and connective tissue. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount, concentration or level. The sample is an in vitro sample, it will be analyzed in vitro and not transferred back into the body.

The terms “nucleic acid” and “nucleic acid molecule” are used synonymously herein and refer to single or double-stranded oligo- or polymers of deoxyribonucleotide or ribonucleotide bases, or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers, In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single- or double-stranded molecules and are composed of naturally occurring nucleotides. The depiction of a single strand of a nucleic acid also defines (at least partially) the sequence of the complementary strand. The nucleic acid may be single or double stranded, or may contain portions of both double and single stranded sequences.

Exemplified, double-stranded nucleic acid molecules can have 3′ or 5′ overhangs and as such are not required or assumed to be completely double-stranded over their entire length. The nucleic acid may be obtained by biological, biochemical or chemical synthesis methods or any of the methods well-known in the art, including but not limited to methods of amplification, and reverse transcription of RNA. The term nucleic acid comprises chromosomes or chromosomal segments, vectors (e.g. expression vectors), expression cassettes, naked DNA or RNA polymer, primers, probes, cDNA, genomic DNA, recombinant DNA, cRNA, mRNA, tRNA, microRNA (miRNA) or small interfering RNA (siRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

The term “complementarity” refers to relationship between two structures following a lock-and-key principle. In nature, complementarity is the base principle of DNA replication and transcription as it is a property shared between two DNA or RNA sequences, such that when they are aligned antiparallel to each other, the nucleotide bases at each position in the sequences will be complementary.

For term “sequence comparison” refers to the process wherein one sequence acts as a reference sequence, to which test sequences are compared. When using a sequence comparison algorithm, test and reference sequences are entered into a computer program, if necessary subsequence coordinates are designated, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters. In a sequence alignment, the term “comparison window” refers to those stretches of contiguous positions of a sequence which are compared to a reference stretch of contiguous positions of a sequence having the same number of positions. The number of contiguous positions selected may range from 10 to 1000, i.e. may comprise 20, 30, 40, 50, 60, 70, 80, 90, 100, 150, 200, 250, 300, 350, 400, 450, 500, 550, 600, 650, 700, 750, 800, 850, 900, 950, or 1000 contiguous positions. Typically, the number of contiguous positions ranges from about 20 to 800 contiguous positions, from about 20 to 600 contiguous positions, from about 50 to 400 contiguous positions, from about 50 to about 200 contiguous positions, from about 100 to about 150 contiguous positions. Methods of alignment of sequences for comparison are well known in the art. Optimal alignment of sequences for comparison can be conducted, for example, by the local algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

The term “at least 90% sequence identity” is used herein with regard to amino acid or nucleotide sequence comparisons. The term “identical” in the context of two or more nucleic acids or polypeptide amino acid sequences, refers to two or more sequences or subsequences that are the same, i.e. comprise the same sequence of nucleotides or amino acids. The term “at least 90% sequence identity” in particular refers to a sequence identity of at least 90%, at least 91%, at least 92%, at least 93%, at least 94%, at least 95%, at least 96%, at least 97%, at least 98%, or at least 99% to the respective amino acid or nucleotide sequence.

The term “at least 90% sequence homology” is used herein with regard to amino acid or nucleotide sequence comparisons. In addition to identical residues (sequence identity) also the percentage of residues conserved with similar physicochemical properties (percent similarity), e.g. leucine and isoleucine, are usually used to “quantify the homology.” The term “at least 90% sequence homology” in particular refers to a sequence homology of at least 90%, at least 91%, at least 92%, at least 93%, at least 94%, at least 95%, at least 96%, at least 97%, at least 98%, or at least 99% to the respective amino acid or nucleotide sequence. Optionally, an amino acid sequence in question and the reference amino acid sequence exhibit the indicated sequence identity or sequence homology over a continuous stretch of 20, 30, 40, 45, 50, 60, 70, 80, 90, 100 or more amino acids or over the entire length of the reference amino acid sequence. Optionally, the nucleic acid sequence in question and the reference nucleic acid sequence exhibit the indicated sequence identity or homology over a continuous stretch of 60, 90, 120, 135, 150, 180, 210, 240, 270, 300, 400, 500, 600, 700, 800, 900, 1000 or more nucleotides or over the entire length of the reference nucleic acid sequence.

The term “recombinant DNA molecule” refers to a molecule which is made by the combination of two otherwise separated segments of DNA sequence accomplished by the artificial manipulation of isolated segments of polynucleotides by genetic engineering techniques or by chemical synthesis. In doing so one may join together polynucleotide segments of desired functions to generate a desired combination of functions. Recombinant DNA techniques for expression of proteins in prokaryotic or lower or higher eukaryotic host cells are well known in the art. They have been described e.g. by Sambrook et al., (1989, Molecular Cloning: A Laboratory Manual).

The terms “vector” and “plasmid” are used interchangeably herein, referring to a protein or a polynucleotide or a mixture thereof which is capable of being introduced or of introducing proteins and/or nucleic acids comprised therein into a cell. Examples of plasmids include but are not limited to plasmids, cosmids, phages, viruses or artificial chromosomes.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Be or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The term “measurement”, “measuring”, “detecting” or “detection” preferably comprises a qualitative, a semi-quantitative or a quantitative measurement. The term “detecting the presence” refers to a qualitative measurement, indicating the presence of absence without any statement to the quantities (e.g. yes or no statement). The term “detecting amount” refers to a quantitative measurement wherein the absolute number is detected (ng). The term “detecting the concentration” refers to a quantitative measurement wherein the amount is determined in relation to a given volume (e.g. ng/ml).

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α,γ, δ, ε, and μ. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜-strands arranged in two ˜-sheets. Typically, the heavy chain of an antibody comprises four Ig domains with three of them being constant (CH domains: CH1. CH2. CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCy1-Cy2-Cy3), whereas the human IgG light chain is composed of two immunoglobulin domains linked from N— to C-terminus in the order VL-CL, being either of the kappa or lambda type (VK-CK or VA.-CA.). Exemplified, the constant chain of human IgG comprises 447 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5^{th}ed. U.S. Department of Health and Human Service, National Institutes of Health, Bethesda, Md. The “EU index as in Kabat” refers to the residue numbering of the human IgG 1EU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The term “antigen (Ag)” is a molecule or molecular structure, which is bound to by an antigen-specific antibody (Ab) or B cell antigen receptor (BCR). The presence of an antigen in the body normally triggers an immune response. In the body, each antibody is specifically produced to match an antigen after cells of the immune system come into contact with it; this allows a precise identification or matching of the antigen and the initiation of a tailored response. In most cases, an antibody can only react to and bind one specific antigen; in some instances, however, antibodies may cross-react and bind more than one antigen. Antigens are normally proteins, peptides (amino acid chains) and polysaccharides (chains of monosaccharides/simple sugars) or combinations thereof.

In diagnostic tests, antigens are often used in serological test to evaluate if a patient has been exposed to a certain pathogen (e.g. virus or bacterium) and has developed antibodies against such pathogen. Typically, these antigens are produced recombinantly and may be linear peptides or more complex folded molecules aiming to represent native antigens.

To resemble native antigens more closely and to obtain a high epitope density, antigens may be generated by polymerizing monomeric antigens by means of chemical crosslinking. There is a wealth of homobifunctional and heterobifunctional crosslinkers that may be used with great advantage and that are well known in the art. Yet, there are some severe drawbacks in the chemically induced polymerization of antigens for use as specifiers in serological assays. For instance, the insertion of crosslinker moieties into antigens may compromise antigenicity by interfering with the native-like conformation or by masking crucial epitopes. Furthermore, the introduction of non-natural tertiary contacts may interfere with the reversibility of protein folding/unfolding, and it may, additionally, be the source of interference problems which have to be overcome by anti-interference strategies in the immunoassay mixture.

A more recent technique is to fuse the antigen of interest to an oligomeric chaperone, thereby conveying high epitope density to the antigen. The advantage of this technology lies in its high reproducibility and in the triple function of the oligomeric chaperone fusion partner: firstly, the chaperone enhances the expression rate of the fusion polypeptide in the host cell (e.g. in E. coli), secondly, the chaperone facilitates the refolding process of the target antigen and enhances its overall solubility and, thirdly, it assembles the target antigen reproducibly into an ordered oligomeric structure.

The term “chaperone” is well-known in the art and refers to protein folding helpers which assist the folding and maintenance of the structural integrity of other proteins. Examples of folding helpers are described in detail in WO 03/000877. Exemplified, chaperones of the peptidyl prolyl isomerase class such as chaperones of the FKBP family can be used for fusion to the antigen variants. Examples of FKBP chaperones suitable as fusion partners are FkpA (aa 26-270, UniProt ID P45523), SlyD (1-165, UniProt ID P0A9K9) and SlpA (2-149, UniProt ID POAEMO). A further chaperone suitable as a fusion partner is Skp (21-161, UniProt ID P0AEU7), a trimeric chaperone from the periplasm of E. coli, not belonging to the FKBP family. It is not always necessary to use the complete sequence of a chaperone. Functional fragments of chaperones (so-called binding-competent modules) which still possess the required abilities and functions may also be used (cf. WO 98/13496).

Antigens may further comprise an “effector group” such as e.g. .a “tag” or a “label”. The term “tag” refers to those effector groups which provide the antigen with the ability to bind to or to be bound to other molecules. Examples of tags include but are not limited to e.g. His tags which are attached to the antigen sequence to allow for its purification. Tag may also include a partner of a bioaffine binding pair which allows the antigen to be bound by the second partner of the binding pair. The term “bioaffine binding pair” refers to two partner molecules (i.e. two partners in one pair) having a strong affinity to bind to each other. Examples of partners of bioaffine binding pairs are a) biotin or biotin analogs/avidin or streptavidin; b) Haptens/anti-hapten antibodies or antibody fragments (e.g. digoxin/anti-digoxin antibodies); c) Saccharides/lectins; d) complementary oligonucleotide sequences (e.g. complementary LNA sequences), and in general e) ligands/receptors.

The term “label” refers to those effector groups which allow for the detection of the antigen. Label include but are not limited to spectroscopic, photochemical, biochemical, immunochemical, or chemical, label. Exemplified, suitable labels include fluorescent dyes, luminescent or electrochemiluminescent complexes (e.g. ruthenium or iridium complexes), electron-dense reagents, and enzymatic label.

A “particle” as used herein means a small, localized object to which can be ascribed a physical property such as volume, mass or average size. Particles may accordingly be of a symmetrical, globular, essentially globular or spherical shape, or be of an irregular, asymmetric shape or form. The size of a particle may vary. The term “microparticle” refers to particles with a diameter in the nanometer and micrometer range.

Microparticles as defined herein above may comprise or consist of any suitable material known to the person skilled in the art, e.g. they may comprise or consist of or essentially consist of inorganic or organic material. Typically, they may comprise or consist of or essentially consist of metal or an alloy of metals, or an organic material, or comprise or consist of or essentially consist of carbohydrate elements. Examples of envisaged material for microparticles include agarose, polystyrene, latex, polyvinyl alcohol, silica and ferromagnetic metals, alloys or composition materials. In one embodiment the microparticles are magnetic or ferromagnetic metals, alloys or compositions. In further embodiments, the material may have specific properties and e.g. be hydrophobic, or hydrophilic. Such microparticles typically are dispersed in aqueous solutions and retain a small negative surface charge keeping the microparticles separated and avoiding non-specific clustering.

In one embodiment of the present invention, the microparticles are paramagnetic microparticles and the separation of such particles in the measurement method according to the present disclosure is facilitated by magnetic forces. Magnetic forces are applied to pull the paramagnetic or magnetic particles out of the solution/suspension and to retain them as desired while liquid of the solution/suspension can be removed and the particles can e.g. be washed.

A “kit” is any manufacture (e.g. a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like In particular, each of the container means comprises one of the separate elements to be used in the method of the first aspect. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

### Embodiments

Currently available ELISA format immunoassays for detecting anti-SARS CoV-2 virus antibodies in patient samples employ Spike protein derived antigens as immunoreactive reagents. However, we find that these assays lack specificity, leading to a fairly high number of false positive results. Surprisingly, by confining the antigen to the Corona nucleocapsid as explained further below, the number of erroneously reactive samples can be considerably reduced while a high sensitivity of the assay is maintained.

Furthermore, all currently ongoing vaccination strategies focus on the development of spike protein based vaccines. Using Spike protein derived antigens for detecting anti-SARS CoV-2 virus antibodies in samples of vaccinated patient allows to determine if the vaccination was successful and the patient developed anti-spike antibodies. However, as it is still not known, how long term effects of vaccination and natural SARS CoV-2 infection will interplay and impact patients, it is important to be able to differentiate whether a patients was exposed to a natural SARS CoV-2 infection or received vaccination in the past. Thus, there is an urgent need for an anti-SARS CoV-2 antibody assay which does not only detect anti-spike-antibodies but which allows to also determine the anti-SARS CoV-2 antibodies directed to other viral proteins.

Accordingly, in a first aspect, the present invention therefore concerns a Corona antigen suitable for detecting antibodies against Corona virus in an isolated biological sample comprising a Corona nucleocapsid specific amino acid sequence according to SEQ ID NO: 1 or a variant thereof. In embodiments, the Corona antigen detects antibodies against Corona virus in an isolated biological sample comprising a Corona nucleocapsid specific amino acid sequence according to SEQ ID NO: 1 or a variant thereof.

In embodiments the antigen comprises no further Corona virus specific amino acid sequences.

In embodiments, the Corona antigen is immunoreactive, i.e. antibodies present in a biological sample bind to said antigen. Accordingly, any peptide derived from Corona nucleocapsid which is not bound by antibodies, is not encompassed.

As shown in FIGS. 1 and 2, the amino acid sequence of SARS CoV-2 exhibits ˜93% sequence homology and ˜90% sequence identity to its closest relative SARS-CoV. The sequence identity and homology to other Coronaviruses is still much lower as shown. Accordingly, already due to the limited sequence identity and homology, the Corona antigen comprising Corona nucleocapsid specific amino acid sequence according to SEQ ID NO: 1 is specific for SARS-CoV and SARS CoV-2detection.

In embodiments, the Corona virus is SARS-CoV or SARS CoV-2 virus, in particular SARS CoV-2 virus. In particular embodiments, the Corona nucleocapsid is a SARS CoV-2 specific nucleocapsid. In particular, the Corona antigen comprising Corona nucleocapsid specific amino acid sequence according to SEQ ID NO: 1 is specific for SARS CoV-2 detection.

In embodiments, the Corona antigen does not immunologically cross-react, i.e. shows only a strongly reduced or completely abolished immunological reactivity, towards antibodies or towards a subset of antibodies raised against the corresponding nucleocapsid antigens of other Corona viruses. In particular, the Corona antigen does not immunologically cross-react with corresponding nucleocapsid antigens from Corona virus strains selected from the group consisting of MERS—CoV, HCoV-NL63, HCoV-229E, HCoV-OC43, HCoV-HKU1. In particular, the Corona antigen does not immunologically cross-react with corresponding nucleocapsid antigens from Corona virus strains selected from the group consisting of SARS-CoV, MERS-CoV, HCoV-NL63, HCoV-229E, HCoV-OC43, HCoV-HKU1.

In embodiments, the Corona antigen is soluble. The Corona antigen is thus, suitable to be used in in vitro assays aiming to detect antibodies against said antigen in isolated biological sample.

The Corona antigen is thus, suitable to be used in in vitro assays aiming to detect anti-Corona antibodies with a high sensitivity and specificity. In embodiments, the sensitivity is >95%, >96%, >97%, >98%, >99%, >99.5%. In particular embodiments, the sensitivity is >99% or >99.5%. In particular embodiments, the sensitivity is 100%. In embodiments, the specificity is >95%, >96%, >97%, >98%, >99%, >99.5%. In particular embodiments, the specificity is >99% or >99.5%. In particular embodiments, the specificity is 99.8%. In particular embodiments, the sensitivity is 100% and the specificity is 99.8%.

In embodiments, the Corona antigen is suitable for detecting or detects antibodies against Corona virus in a fluid sample. In particular embodiments, the sample is a human sample, in particular in a human body fluid sample. In particular embodiments, the sample is a human blood or urine sample. In particular embodiments the sample is a human whole blood, plasma, or serum sample.

In embodiments, the Corona antigen is a linear antigen or in its native state. In particular embodiments, the Corona nucleocapsid specific amino acid sequence comprised in the Corona antigen is folded in its native state.

In embodiments, the variants of the Corona nucleocapsid specific amino acid sequences of SEQ ID NO:1 are encompassed. These variants are easily created by a person skilled in the art by conservative or homologous substitutions of the disclosed amino acid sequences (such as e.g. substitutions of a cysteine by alanine or serine).

In embodiments, the variant exhibits modifications to its amino acid sequence, in particular selected from the group consisting of amino acid exchanges, deletions or insertions compared to the amino acid sequence of SEQ ID NO: 1.

In embodiments, amino acid are C- or N-terminal deleted or inserted at one end or at both ends by 1 to 10 amino acids, in an embodiment by 1 to 5 amino acids. In particular, a variant may be an isoform which shows the most prevalent protein isoform. In one embodiment, such a substantially similar protein has a sequence homology to SEQ ID NO: 1 of at least 95%, in particular of at least 96%, in particular of at least 97%, in particular of at least 98%, in particular of at least 99%.

In embodiments, Corona nucleocapsid variant comprises an amino acid sequence according to SEQ ID NO: 8, SEQ ID NO: 10, SEQ ID NO: 12, or SEQ ID NO: 14.

In embodiments, the variant comprises post-translationally modifications, in particular selected from the group consisting of glycosylation or phosphorylation.

It is understood, that such variant classifies as a Corona nucleocapsid variant, i.e. is able to bind and detect anti-Corona antibodies present in an isolated sample.

In embodiments, the overall three-dimensional structure of the Corona nucleocapsid remains unaltered, so that epitopes that were previously (i.e. in the wild type) accessible for binding to antibodies are still accessible in the variant.

In embodiments, the Corona antigen further comprises at least one chaperone. Accordingly, the Corona antigen comprises the Corona nucleocapsid specific amino acid sequences of SEQ ID NO:1 as described above or below, and the amino acid sequence of a chaperone.

In particular embodiments, the Corona antigen comprises 2 chaperones. In embodiments, said chaperone is selected from the group consisting of SlyD, SipA, FkpA, and Skp. In particular embodiments, the chaperone is SlyD, in particular having an amino acid sequence given in accession no: UniProt ID P0A9K9.

In particular embodiments, the Corona antigen comprises a Corona nucleocapsid specific amino acid sequence according to SEQ ID NO: 1, SEQ ID NO: 8, SEQ ID NO: 10, SEQ ID NO: 12, or SEQ ID NO: 14, and one SlyD chaperone. In particular embodiments, the Corona antigen comprises a Corona nucleocapsid specific amino acid sequence according to SEQ ID NO: 1, SEQ ID NO: 8, SEQ ID NO: 10, SEQ ID NO: 12, or SEQ ID NO: 14, and two SlyD chaperones.

The fusion of two chaperone results in a higher solubility of the resulting antigen.

In embodiments, the chaperone is fused to the Corona nucleocapsid specific amino acid sequence at the N— and/or— C-terminus of the nucleocapsid, in particular to the N-terminus of the nucleocapsid. Accordingly, in particular embodiments, the Corona antigen comprises one SlyD chaperone N-terminal of the Corona nucleocapsid specific amino acid sequence. In particular embodiments, the Corona antigen comprises two SlyD chaperone N-terminal of the Corona nucleocapsid specific amino acid sequence. In embodiments, the Corona antigen comprises one SlyD chaperone N-terminal of the Corona nucleocapsid specific amino acid sequence and one SlyD chaperone C-terminal of the Corona nucleocapsid specific amino acid sequence.

In embodiments, the Corona antigen further comprises linker sequences. These sequences are not specific for anti-Corona virus antibodies and are not be recognized in an in vitro diagnostic immunoassay. In particular, the Corona antigen comprises linker sequences between the sequence of the Corona nucleocapsid and the one or more chaperones. In particular embodiments, the linker is a Gly-rich linker. In particular embodiments, the linker has the sequence as indicated in SEQ ID NO: 7.

In particular embodiments, the Corona antigen comprises an amino acid sequence according to SEQ ID NO: 2. In embodiments, the Corona antigen does not comprise any further amino acid sequences. In particular embodiments, the Corona antigen consists of amino acid sequence according to SEQ ID NO: 2.

In particular embodiments, the Corona antigen comprises an amino acid sequence according to SEQ ID NO: 3. In embodiments, the Corona antigen does not comprise any further amino acid sequences. In particular embodiments, the Corona antigen consists of SEQ ID NO: 3.

In embodiments, the Corona antigen comprises an amino acid sequence according to SEQ ID NO: 9, SEQ ID NO: 11, SEQ ID NO: 13, or SEQ ID NO: 15. In embodiments, the Corona antigen does not comprise any further amino acid sequences. In particular embodiments, the Corona antigen consists of SEQ ID NO: 9, SEQ ID NO: 11, SEQ ID NO: 13, or SEQ ID NO: 15 It is understood, that a Corona antigen consisting of SEQ ID NO: 2, SEQ ID NO: 3, SEQ ID NO: 9, SEQ ID NO: 11, SEQ ID NO: 13, or SEQ ID NO: 15 does not comprise any additional amino acid sequences, but may still comprise other chemical molecules, such as e.g. labels and/or tags.

In particular embodiments, the sequence homology to SEQ ID NO: 1, SEQ ID NO: 2, or SEQ ID NO: 3 is at least 96%, at least 97%, at least 98%, or at least 99%. In particular embodiments, the sequence homology to SEQ ID NO: 1, SEQ ID NO: 2, or SEQ ID NO: 3 is at least 98%.

In particular embodiments, the sequence homology to SEQ ID NO:8, SEQ ID NO: 9, SEQ ID NO: 10, or SEQ ID NO: 11. SEQ ID NO: 12, SEQ ID NO: 13, SEQ ID NO: 14, or SEQ ID NO: 15, is at least 96%, at least 97%, at least 98%, or at least 99%. In particular embodiments, the sequence homology to SEQ ID NO:8, SEQ ID NO: 9, SEQ ID NO: 10, or SEQ ID NO: 11. SEQ ID NO: 12, SEQ ID NO: 13, SEQ ID NO: 14, or SEQ ID NO: 15 is at least 98%.

In embodiments, the Corona antigen further comprises a tag or a label. Accordingly, the Corona antigen comprises the Corona nucleocapsid specific amino acid sequences of SEQ ID NO: 1, SEQ ID NO: 8, SEQ ID NO: 10, SEQ ID NO: 12, or SEQ ID NO: 14 as described above or below, and a tag and/or a label, and optionally the amino acid sequence of one or more chaperones.

In particular embodiments, the tag allows to bind the Corona antigen directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the Corona antigen. In particular embodiments, the Corona specific nucleocapsid sequence is labeled. In embodiments wherein at least one chaperone is present in the antigen, the Corona specific nucleocapsid sequence is labeled or the at least one chaperone is labeled, or both are labeled. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, the label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In a second aspect, the present invention relates to a composition comprising a Corona antigen suitable for detecting antibodies against Corona virus in an isolated biological sample comprising a Corona nucleocapsid specific amino acid sequence according to SEQ ID NO: 1 or a variant thereof. In embodiments, the Corona antigen detects antibodies against Corona virus in an isolated biological sample comprising a Corona nucleocapsid specific amino acid sequence according to SEQ ID NO: 1 or a variant thereof.

In embodiments, the Corona antigen comprises no further Corona virus specific amino acid sequences.

In embodiments, the Corona antigen is immunoreactive, i.e. antibodies present in a biological sample bind to said antigen. Accordingly, any peptide derived from Corona nucleocapsid which is not bound by antibodies, is not encompassed.

As shown in FIGS. 1 and 2, the amino acid sequence of SARS CoV-2 exhibits ˜93% sequence homology and ˜90% sequence identity to its closest relative SARS-CoV. The sequence identity and homology to other Coronaviruses is still much lower as shown. Accordingly, already due to the limited sequence identity and homology, the Corona antigen comprising Corona nucleocapsid specific amino acid sequence according to SEQ ID NO: 1 is specific for SARS-CoV and SARS CoV-2 detection.

In embodiments, the Corona virus is SARS-CoV or SARS CoV-2 virus, in particular SARS CoV-2 virus. In particular embodiments, the Corona nucleocapsid is a SARS CoV-2 specific nucleocapsid. In particular, the Corona antigen comprising Corona nucleocapsid specific amino acid sequence according to SEQ ID NO: 1 is specific for SARS CoV-2 detection.

In embodiments, the Corona antigen does not immunologically cross-react, i.e. shows only a strongly reduced or completely abolished immunological reactivity, towards antibodies or towards a subset of antibodies raised against the corresponding nucleocapsid antigens of other Corona viruses. In particular, the Corona antigen does not immunologically cross-react with corresponding nucleocapsid antigens from Corona virus strains selected from the group consisting of MERS—CoV, HCoV-NL63, HCoV-229E, HCoV-OC43, HCoV-HKU1. In particular, the Corona antigen does not immunologically cross-react with corresponding nucleocapsid antigens from Corona virus strains selected from the group consisting of SARS-CoV, MERS-CoV, HCoV-NL63, HCoV-229E, HCoV-OC43, HCoV-HKU1.

In embodiments, the Corona antigen is soluble. The Corona antigen is thus, suitable to be used in in vitro assays aiming to detect antibodies against said antigen in isolated biological sample.

The Corona antigen is thus, suitable to be used in in vitro assays aiming to detect anti-Corona antibodies with a high sensitivity and specificity. In embodiments, the sensitivity is >95%, >96%, >97%, >98%, >99%, >99.5%. In particular embodiments, the sensitivity is >99% or >99.5%. In particular embodiments, the sensitivity is 100%. In embodiments, the specificity is >95%, >96%, >97%, >98%, >99%, >99.5%. In particular embodiments, the specificity is >99% or >99.5%. In particular embodiments, the specificity is 99.8%. In particular embodiments, the sensitivity is 100% and the specificity is 99.8%.

In embodiments, the Corona antigen is suitable for detecting or detects antibodies against Corona virus in a fluid sample. In particular embodiments, the sample is a human sample, in particular in a human body fluid sample. In particular embodiments, the sample is a human blood or urine sample. In particular embodiments the sample is a human whole blood, plasma, or serum sample.

In embodiments, the Corona antigen is a linear antigen or in its native state. In particular embodiments, the Corona nucleocapsid specific amino acid sequence comprised in the Corona antigen is folded in its native state.

In embodiments, the variants of the Corona nucleocapsid specific amino acid sequences of SEQ ID NO:1 are encompassed. These variants may easily be created by a person skilled in the art by conservative or homologous substitutions of the disclosed amino acid sequences (such as e.g. substitutions of a cysteine by alanine or serine). In embodiments, the variant exhibits modifications to its amino acid sequence, in particular selected from the group consisting of amino acid exchanges, deletions or insertions compared to the amino acid sequence of SEQ ID NO: 1.

In embodiments, amino acid are C- or N-terminal deleted or inserted at one end or at both ends by 1 to 10 amino acids, in an embodiment by 1 to 5 amino acids. In particular, a variant may be an isoform which shows the most prevalent protein isoform. In one embodiment, such a substantially similar protein has a sequence homology to SEQ ID NO: 1 of at least 95%, in particular of at least 96%, in particular of at least 97%, in particular of at least 98%, in particular of at least 99%.

In embodiments, Corona nucleocapsid variant comprises an amino acid sequence according to SEQ ID NO: 8, SEQ ID NO: 10, SEQ ID NO: 12, or SEQ ID NO: 14.

In embodiments, the variant comprises post-translationally modifications, in particular selected from the group consisting of glycosylation or phosphorylation.

It is understood, that such variant classifies as a Corona nucleocapsid variant, i.e. is able to bind and detect anti-Corona antibodies present in an isolated sample.

In embodiments, the overall three-dimensional structure of the Corona nucleocapsid remains unaltered, so that epitopes that were previously (i.e. in the wild type) accessible for binding to antibodies are still accessible in the variant.

In embodiments, the Corona antigen further comprises at least one chaperone. Accordingly, the Corona antigen comprises the Corona nucleocapsid specific amino acid sequences of SEQ ID NO: 1 as described above or below, and the amino acid sequence of a chaperone.

In particular embodiments, the Corona antigen comprises 2 chaperones. In embodiments, said chaperone is selected from the group consisting of SlyD, SlpA, FkpA, and Skp. In particular embodiments, the chaperone is SlyD, in particular having an amino acid sequence given in accession no: UniProt ID POA9K9.

In particular embodiments, the Corona antigen comprises a Corona nucleocapsid specific amino acid sequence according to SEQ ID NO: 1, SEQ ID NO: 8, SEQ ID NO: 10, SEQ ID NO: 12, or SEQ ID NO: 14, and one SlyD chaperone. In particular embodiments, the Corona antigen comprises a Corona nucleocapsid specific amino acid sequence according to SEQ ID NO: 1, SEQ ID NO: 8, SEQ ID NO: 10, SEQ ID NO: 12, or SEQ ID NO: 14, and two SlyD chaperones.

The fusion of two chaperone results in a higher solubility of the resulting antigen.

In embodiments, the chaperone is fused to the Corona nucleocapsid specific amino acid sequence at the N— and/or— C-terminus of the nucleocapsid, in particular to the N-terminus of the nucleocapsid. Accordingly, in particular embodiments, the Corona antigen comprises one SlyD chaperone N-terminal of the Corona nucleocapsid specific amino acid sequence. In particular embodiments, the Corona antigen comprises two SlyD chaperone N-terminal of the Corona nucleocapsid specific amino acid sequence. In embodiments, the Corona antigen comprises one SlyD chaperone N-terminal of the Corona nucleocapsid specific amino acid sequence and one SlyD chaperone C-terminal of the Corona nucleocapsid specific amino acid sequence.

In embodiments, the Corona antigen further comprises linker sequences. These sequences are not specific for anti-Corona virus antibodies and are not be recognized in an in vitro diagnostic immunoassay. In particular, the Corona antigen comprises linker sequences between the sequence of the Corona nucleocapsid and the one or more chaperones. In particular embodiments, the linker is a Gly-rich linker. In particular embodiments, the linker has the sequence as indicated in SEQ ID NO: 7.

In particular embodiments, the Corona antigen comprises an amino acid sequence according to SEQ ID NO: 2. In embodiments, the Corona antigen does not comprise any further amino acid sequences. In particular embodiments, the Corona antigen consists of amino acid sequence according to SEQ ID NO: 2.

In particular embodiments, the Corona antigen comprises an amino acid sequence according to SEQ ID NO: 3. In embodiments, the Corona antigen does not comprise any further amino acid sequences. In particular embodiments, the Corona antigen consists of SEQ ID NO: 3.

In embodiments, the Corona antigen comprises an amino acid sequence according to SEQ ID NO: 9, SEQ ID NO: 11, SEQ ID NO: 13, or SEQ ID NO: 15. In embodiments, the Corona antigen does not comprise any further amino acid sequences. In particular embodiments, the Corona antigen consists of SEQ ID NO: 9, SEQ ID NO: 11, SEQ ID NO: 13, or SEQ ID NO: 15 It is understood, that a Corona antigen consisting of SEQ ID NO: 2 or SEQ ID NO: 3 does not comprise any additional amino acid sequences, but may still comprise other chemical molecules, such as e.g. labels and/or tags.

In particular embodiments, the sequence homology to SEQ ID NO: 1, SEQ ID NO: 2, or SEQ ID NO: 3 is at least 96%, at least 97%, at least 98%, or at least 99%. In particular embodiments, the sequence homology to SEQ ID NO: 1, SEQ ID NO: 2, or SEQ ID NO: 3 is at least 98%.

In particular embodiments, the sequence homology to SEQ ID NO:8, SEQ ID NO: 9, SEQ ID NO: 10, or SEQ ID NO: 11. SEQ ID NO: 12, SEQ ID NO: 13, SEQ ID NO: 14, or SEQ ID NO: 15, is at least 96%, at least 97%, at least 98%, or at least 99%. In particular embodiments, the sequence homology to SEQ ID NO:8, SEQ ID NO: 9, SEQ ID NO: 10, or SEQ ID NO: 11. SEQ ID NO: 12, SEQ ID NO: 13, SEQ ID NO: 14, or SEQ ID NO: 15 is at least 98%.

In embodiments, the Corona antigen further comprises a tag or a label. In particular embodiments, the Corona specific nucleocapsid sequence is labeled. In embodiments wherein at least one chaperone is present in the antigen, the Corona specific nucleocapsid sequence is labeled or the at least one chaperone is labeled, or both are labeled.

Accordingly, the Corona antigen comprises the Corona nucleocapsid specific amino acid sequences of SEQ ID NO: 1, SEQ ID NO: 8, SEQ ID NO: 10, SEQ ID NO: 12, or SEQ ID NO: 14 as described above or below, and a tag and/or a label, and optionally the amino acid sequence of one or more chaperones.

In particular embodiments, the tag allows to bind the antigen directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the antigen. In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In embodiments, the composition comprises one or more additional Corona antigens. In particular embodiments, the composition comprises 1, 2 or 3 additional antigens. In particular embodiments, the composition comprises one or more additional Corona antigens comprising amino acid sequences of the E-protein, the M-protein, and/or the S-protein, or parts thereof. In particular embodiments, the composition comprises an additional Corona antigen comprising the amino acid sequences of the S-protein or parts thereof (e.g. the receptor binding domain of the S-protein).

In particular embodiments, the additional Corona antigens are immunoreactive, i.e. antibodies present in a biological sample bind to said antigen. Accordingly, any peptide derived from Corona which is not bound by anti-Corona antibodies, is not encompassed.

In embodiments, the additional Corona antigen does not immunologically cross-react, i.e. shows only a strongly reduced or completely abolished immunological reactivity, towards antibodies or towards a subset of antibodies raised against the corresponding antigens of other Corona viruses. In particular, additional Corona antigen does not immunologically cross-react with corresponding antigens from Corona virus strains selected from the group consisting of MERS—CoV, HCoV-NL63, HCoV-229E, HCoV-OC43, HCoV-HKU1. In particular, the additional Corona antigen does not immunologically cross-react with corresponding antigens from Corona virus strains selected from the group consisting of SARS-CoV, MERS-CoV, HCoV-NL63, HCoV-229E, HCoV-OC43, HCoV-HKU1.

In embodiments, the additional Corona antigen is soluble. The antigen is thus, suitable to be used in in vitro assays aiming to detect antibodies against said antigen in isolated biological sample.

In a third aspect, the present invention relates to a method of producing a Corona antigen specific for Corona virus nucleocapsid, said method comprising the steps of
a) culturing host cells transformed with an expression vector comprising operably linked a recombinant DNA molecule encoding a Corona antigen as describes above for the first aspect of the present invention,
b) expression of said polypeptide and
c) purification of said polypeptide.

Optionally, as an additional step d), functional solubilization needs to be carried out so that the Corona nucleocapsid antigen is brought into a soluble and immunoreactive conformation by means of refolding techniques known in the art.

In particular embodiments, the host cells are E. coli cells, CHO cells, or HEK cells. In particular embodiments, the host cells are E. coli cells.

In embodiments, wherein the antigen comprises the Corona nucleocapsid and one or more chaperones, the recombinant DNA molecules according to the invention may also contain sequences encoding linker peptides of 5 to 100 amino acid residues in between the Corona antigen. Such a linker sequence may for example harbor a proteolytic cleavage site. In an embodiment, the addition of non-Corona-specific linker or peptidic fusion amino acid sequences to the Corona nucleocapsid is possible as these sequences are not specific for anti-Corona virus antibodies and would not be recognized in an in vitro diagnostic immunoassay.

In particular embodiments, the recombinant DNA molecule comprising a sequence according to SEQ ID NO: 4.

In particular embodiments, the recombinant DNA molecule comprising a sequence according to SEQ ID NO: 5.

In particular embodiments, the recombinant DNA molecule comprising a sequence according to SEQ ID NO: 6.

In a fourth aspect, the present invention relates to a method for detecting antibodies specific for Corona virus in an isolated biological sample, wherein a Corona antigen according to the first aspect of the present invention, the composition of the second aspect of the present invention, or a Corona antigen obtained by a method according to the third aspect of the present invention, is used as a capture reagent and/or as a binding partner for said anti-Corona virus antibodies.

In a fifth, aspect, the present invention relates to a method for detecting antibodies specific for Corona virus in an isolated biological sample, said method comprising
a) forming an immunoreaction mixture by admixing the isolated biological sample with a Corona antigen or a composition comprising a Corona antigen,
b) maintaining said immunoreaction admixture for a time period sufficient for allowing antibodies present in the isolated biological sample against said Corona antigen to immunoreact with said Corona antigen to form an immunoreaction product; and
c) detecting the presence, amount, and/or the concentration of any of said immunoreaction product.

In embodiments, the method is an in vitro method. In embodiments, the method exhibits a high sensitivity and specificity. In embodiments, the sensitivity is >95%, >96%, >97%, >98%, >99%, >99.5%. In particular embodiments, the sensitivity is >99% or >99.5%. In particular embodiments, the sensitivity is 100%. In embodiments, the specificity is >95%, >96%, >97%, >98%, >99%, >99.5%. In particular embodiments, the specificity is >99% or >99.5%. In particular embodiments, the specificity is 99.8%. In particular embodiments, the sensitivity is 100% and the specificity is 99.8%.

In embodiments, the antibodies detected by the method of the present invention are anti-Corona virus antibodies of the IgG, the IgM, or the IgA subclass, or of all three subclasses in the same immunoassay.

In embodiments, the antibodies detected are directed against the nucleocapsid of the Corona virus, in particular antibodies directed against the nucleocapsid of SARS-CoV or SARS CoV-2virus. In particular embodiments, the antibodies detected are directed against the nucleocapsid of SARS CoV-2virus.

In embodiments, the isolated biological sample in which the Corona specific antibodies are detected, is a human sample, in particular in a human body fluid sample. In particular embodiments, the sample is a human blood or urine sample. In particular embodiments the sample is a human whole blood, plasma, or serum sample. In particular embodiments the sample is a venous or capillary human whole blood, plasma, or serum sample.

In embodiments, the Corona antigen admixed to the isolated biological sample in step a) comprises a Corona nucleocapsid specific amino acid sequence according to SEQ ID NO: 1 or a variant thereof. In embodiments the Corona antigen comprises no further Corona virus specific amino acid sequences.

In embodiments, the Corona antigen is immunoreactive, i.e. antibodies present in a biological sample bind to said antigen. Accordingly, any peptide derived from Corona nucleocapsid which is not bound by antibodies, is not encompassed.

As shown in FIGS. 1 and 2, the amino acid sequence of SARS CoV-2 exhibits ˜93% sequence homology and ˜90% sequence identity to its closest relative SARS-CoV. The sequence identity and homology to other Coronaviruses is still much lower as shown. Accordingly, already due to the limited sequence identity and homology, the Corona antigen comprising Corona nucleocapsid specific amino acid sequence according to SEQ ID NO: 1 is specific for SARS-CoV and SARS CoV-2 detection.

In embodiments, the Corona virus is SARS-CoV or SARS CoV-2 virus, in particular SARS CoV-2virus. In particular embodiments, the Corona nucleocapsid is a SARS CoV-2 specific nucleocapsid. In particular, the Corona antigen comprising Corona nucleocapsid specific amino acid sequence according to SEQ ID NO: 1 is specific for SARS CoV-2 detection.

In embodiments, the Corona antigen does not immunologically cross-react, i.e. shows only a strongly reduced or completely abolished immunological reactivity, towards antibodies or towards a subset of antibodies raised against the corresponding nucleocapsid antigens of other Corona viruses. In particular, the Corona antigen does not immunologically cross-react with corresponding nucleocapsid antigens from Corona virus strains selected from the group consisting of MERS—CoV, HCoV-NL63, HCoV-229E, HCoV-OC43, HCoV-HKU1. In particular, the Corona antigen does not immunologically cross-react with corresponding nucleocapsid antigens from Corona virus strains selected from the group consisting of SARS-CoV, MERS-CoV, HCoV-NL63, HCoV-229E, HCoV-OC43, HCoV-HKU1.

In embodiments, the Corona antigen is soluble. The Corona antigen is thus, suitable to be used in in vitro assays aiming to detect antibodies against said antigen in isolated biological sample.

The Corona antigen is thus, suitable to be used in in vitro assays aiming to detect anti-Corona antibodies with a high sensitivity and specificity. In embodiments, the sensitivity is >95%, >96%, >97%, >98%, >99%, >99.5%. In particular embodiments, the sensitivity is >99% or >99.5%. In particular embodiments, the sensitivity is 100%. In embodiments, the specificity is >95%, >96%, >97%, >98%, >99%, >99.5%. In particular embodiments, the specificity is >99% or >99.5%. In particular embodiments, the specificity is 99.8%. In particular embodiments, the sensitivity is 100% and the specificity is 99.8%.

In embodiments, the Corona antigen is soluble. The antigen is thus, suitable to be used in the present in vitro method.

In embodiments, the Corona antigen is a linear antigen or in its native state. In particular embodiments, the Corona nucleocapsid specific amino acid sequence comprised in the antigen is folded in its native state.

In embodiments, the variants of the Corona nucleocapsid specific amino acid sequences of SEQ ID NO: 1 are encompassed. These variants may easily be created by a person skilled in the art by conservative or homologous substitutions of the disclosed amino acid sequences (such as e.g. substitutions of a cysteine by alanine or serine). In embodiments, the variant exhibits modifications to its amino acid sequence, in particular selected from the group consisting of amino acid exchanges, deletions or insertions compared to the amino acid sequence of SEQ ID NO: 1.

In embodiments, amino acid are C- or N-terminal deleted or inserted at one end or at both ends by 1 to 10 amino acids, in an embodiment by 1 to 5 amino acids. In particular, a variant may be an isoform which shows the most prevalent protein isoform. In one embodiment, such a substantially similar protein has a sequence homology to SEQ ID NO: 1 of at least 95%, in particular of at least 96%, in particular of at least 97%, in particular of at least 98%, in particular of at least 99%.

In embodiments, Corona nucleocapsid variant comprises an amino acid sequence according to SEQ ID NO: 8, SEQ ID NO: 10, SEQ ID NO: 12, or SEQ ID NO: 14.

In embodiments, the variant comprises post-translationally modifications, in particular selected from the group consisting of glycosylation or phosphorylation.

It is understood, that such variant classifies as a Corona nucleocapsid variant, i.e. is able to bind and detect anti-Corona antibodies present in an isolated sample.

In embodiments, the overall three-dimensional structure of the Corona nucleocapsid remains unaltered, so that epitopes that were previously (i.e. in the wild type) accessible for binding to antibodies are still accessible in the variant.

In embodiments, the Corona antigen further comprises at least one chaperone. Accordingly, the Corona antigen comprises the Corona nucleocapsid specific amino acid sequences of SEQ ID NO: 1 as described above or below, and the amino acid sequence of a chaperone.

In particular embodiments, the Corona antigen comprises 2 chaperones. In embodiments, said chaperone is selected from the group consisting of SlyD, SlpA, FkpA and Skp. In particular embodiments, the chaperone is Sly D, in particular having an amino acid sequence given in accession no: UniProt ID P0A9K9.

In particular embodiments, the Corona antigen comprises a Corona nucleocapsid specific amino acid sequence according to SEQ ID NO: 1, SEQ ID NO: 8, SEQ ID NO: 10, SEQ ID NO: 12, or SEQ ID NO: 14, and one SlyD chaperone. In particular embodiments, the Corona antigen comprises a Corona nucleocapsid specific amino acid sequence according to SEQ ID NO: 1, SEQ ID NO: 8, SEQ ID NO: 10, SEQ ID NO: 12, or SEQ ID NO: 14, and two SlyD chaperones.

The fusion of two chaperone results in a higher solubility of the resulting antigen.

In embodiments, the chaperone is fused to the Corona nucleocapsid specific amino acid sequence at the N— and/or— C-terminus of the nucleocapsid, in particular to the N-terminus of the nucleocapsid. Accordingly, in particular embodiments, the Corona antigen comprises one SlyD chaperone N-terminal of the Corona nucleocapsid specific amino acid sequence. In particular embodiments, the Corona antigen comprises two SlyD chaperone N-terminal of the Corona nucleocapsid specific amino acid sequence. In embodiments, the Corona antigen comprises one SlyD chaperone N-terminal of the Corona nucleocapsid specific amino acid sequence and one SlyD chaperone C-terminal of the Corona nucleocapsid specific amino acid sequence.

In embodiments, the Corona antigen further comprises linker sequences. These sequences are not specific for anti-Corona virus antibodies and are not be recognized in an in vitro diagnostic immunoassay. In particular, the Corona antigen comprises linker sequences between the sequence of the Corona nucleocapsid and the one or more chaperones. In particular embodiments, the linker is a Gly-rich linker. In particular embodiments, the linker has the sequence as indicated in SEQ ID NO: 7.

In particular embodiments, the Corona antigen comprises an amino acid sequence according to SEQ ID NO: 2. In embodiments, the Corona antigen does not comprise any further amino acid sequences. In particular embodiments, the Corona antigen consists of amino acid sequence according to SEQ ID NO: 2.

In particular embodiments, the Corona antigen comprises an amino acid sequence according to SEQ ID NO: 3. In embodiments, the Corona antigen does not comprise any further amino acid sequences. In particular embodiments, the Corona antigen consists of SEQ ID NO: 3.

In embodiments, the Corona antigen comprises an amino acid sequence according to SEQ ID NO: 9, SEQ ID NO: 11, SEQ ID NO: 13, or SEQ ID NO: 15. In embodiments, the Corona antigen does not comprise any further amino acid sequences. In particular embodiments, the Corona antigen consists of SEQ ID NO: 9, SEQ ID NO: 11, SEQ ID NO: 13, or SEQ ID NO: 15 It is understood, that a Corona antigen consisting of SEQ ID NO: 2 or SEQ ID NO: 3 does not comprise any additional amino acid sequences, but may still comprise other chemical molecules, such as e.g. labels and/or tags.

In particular embodiments, the sequence homology to SEQ ID NO: 1, SEQ ID NO: 2, or SEQ ID NO: 3 is at least 96%, at least 97%, at least 98%, or at least 99%. In particular embodiments, the sequence homology to SEQ ID NO: 1, SEQ ID NO: 2, or SEQ ID NO: 3 is at least 98%.

In particular embodiments, the sequence homology to SEQ ID NO: 8, SEQ ID NO: 9, SEQ ID NO: 10, or SEQ ID NO: 11. SEQ ID NO: 12, SEQ ID NO: 13, SEQ ID NO: 14, or SEQ ID NO: 15, is at least 96%, at least 97%, at least 98%, or at least 99%. In particular embodiments, the sequence homology to SEQ ID NO: 8, SEQ ID NO: 9, SEQ ID NO: 10, or SEQ ID NO: 11. SEQ ID NO: 12, SEQ ID NO: 13, SEQ ID NO: 14, or SEQ ID NO: 15 is at least 98%.

In embodiments, the Corona antigen further comprises a tag or a label. Accordingly, the Corona antigen comprises the Corona nucleocapsid specific amino acid sequences of SEQ ID NO: 1, SEQ ID NO: 8, SEQ ID NO: 10, SEQ ID NO: 12, or SEQ ID NO: 14 as described above or below, and a tag and/or a label, and optionally the amino acid sequence of one or more chaperones.

In particular embodiments, the tag allows to bind the antigen directly or indirectly to a solid phase. In particular embodiments, the tag is a partner of a bioaffine binding pair. In particular embodiments, the tag is selected from the group consisting of biotin, digoxin, hapten, or complementary oligonucleotide sequences (in particular complementary LNA sequences). In particular embodiments, the tag is biotin.

In particular embodiments, the label allows for the detection of the Corona antigen. In particular embodiments, the Corona specific nucleocapsid sequence is labeled. In embodiments wherein at least one chaperone is present in the antigen, the Corona specific nucleocapsid sequence is labeled or the at least one chaperone is labeled, or both are labeled.

In particular embodiments, the label is an electrochemiluminescent ruthenium or iridium complex. In particular embodiments, the electrochemiluminescent ruthenium complex is a negatively charged electrochemiluminescent ruthenium complex. In particular embodiments, label is a negatively charged electrochemiluminescent ruthenium complex which is present in the antigen with a stoichiometry of 1:1 to 15:1. In particular embodiments the stoichiometry is 2:1, 2.5:1, 3:1, 5:1, 10:1, or 15:1.

In embodiments, the method comprises the additional step of adding a solid phase to the immunoreaction mixture. In embodiments, the solid phases is a Solid Phase Extraction (SPE) cartridges, or beads. In particular embodiments, the solid phase comprises or consists of particles. In embodiments, the particles are non-magnetic, magnetic, or paramagnetic. In embodiments, the particles are coated. The coating may differ depending on the use intended, i.e. on the intended capture molecule. It is well-known to the skilled person which coating is suitable for which analyte. The particles may be made of various different materials. The beads may have various sizes and comprise a surface with or without pores.

In particular embodiments, the particles are microparticles. In embodiments, the microparticles have a diameter of 50 nanometers to 20 micrometers. In embodiments, the microparticles have a diameter of between 100 nm and 10 μm. In embodiments, the microparticles have a diameter of 200 nm to 5 μm, in particular of 750 nm to 5 μm, in particular of 750 nm to 2 μm. In particular embodiments the microparticles are magnetic or paramagnetic. In particular, the microparticles are paramagnetic.

In embodiments, the solid phase is added either before the addition of the sample to said antigens or after the immunoreaction admixture is formed. Accordingly, the addition of the solid phase may take place in step a) of the present method, in step b) or the present method, or after step b) of the present method.

In embodiments, the performed method is an immunoassay for detecting anti-Corona antibodies in an isolated biological sample. Immunoassays for detection of antibodies are well known in the art, and so are methods for carrying out such assays and practical applications and procedures. The Corona nucleocapsid antigens according to the invention can be used to improve assays for the detection of anti-Corona antibodies independently of the labels used and independently of the mode of detection (e.g., radioisotope assay, enzyme immunoassay, electrochemiluminescence assay, etc.) or the assay principle (e.g., test strip assay, sandwich assay, indirect test concept or homogenous assay, etc.).

In embodiments, the performed method is an immunoassay for detecting anti-Corona antibodies in an isolated sample according to the so-called double antigen sandwich concept (DAGS). Sometimes this assay concept is also termed double antigen bridge concept, because the two antigens are bridged by an antibody analyte. In such an assay the ability of an antibody to bind at least two different molecules of a given antigen with its two (IgG, IgE), four (IgA) or ten (IgM) paratopes is utilized.

In embodiments, an immunoassay for the determination of anti-Corona antibodies according to the DAGS format is carried out by incubating a sample containing the anti-Corona antibodies with two different Corona antigens, i.e. a first (“capture”) Corona antigen and a second Corona virus (“detection”) antigen, wherein each of the two antigens is bound specifically by anti-Corona antibodies.

In embodiments, the structure of the “capture antigen” and the “detection antigen” are immunologically cross-reactive. The essential requirement for performing the present method is that the relevant epitope or the relevant epitopes are present on both antigens. Accordingly, both antigens comprise a cornoa nucleocapsid specific amino acid sequence as described above or below. In embodiments, the two antigens comprise the same or different fusion moieties (e.g. SlyD fused to Corona nucleocapsid specific antigen tagged to be bound by a solid phase, and, e.g., FkpA fused to Corona nucleocapsid specific antigen labeled to be detected) as such variations significantly alleviate the problem of non-specific binding and thus mitigate the risk of false-positive results.

In embodiments, the first antigen can be bound directly or indirectly to a solid phase and usually carries an effector group which is part of a bioaffine binding pair. In particular embodiments, the first antigen is conjugated to biotin and the complementary solid phase is coated with either avidin or streptavidin. In embodiments, the second antigen carries a label that confers specific detectability to this antigen molecule, either alone or in complex with other molecules. In particular embodiments, the second antigen carries a ruthenium complex label.

Thus, in step b) of the present method, an immunoreaction admixture is formed comprising the first antigen, the sample antibody and the second antigen.

This ternary complex consisting of analyte antibody sandwiched in between two antigen molecules is termed immunocomplex or immunoreaction product.

In embodiments, the method may comprise the additional step of separating the liquid phase from the solid phase.

Accordingly, in embodiments, the method for detecting antibodies specific for Corona virus in an isolated sample comprises
a) adding to said sample a first Corona antigen which can be bound directly or indirectly to a solid phase and carries an effector group which is part of a bioaffine binding pair, and a second Corona antigen which carries a detectable label, wherein said first and second Corona antigens bind specifically to said anti-Corona antibodies
b) forming an immunoreaction admixture comprising the first antigen, the sample antibody and the second antigen wherein a solid phase carrying the corresponding effector group of said bioaffine binding pair is added before, during or after forming the immunoreaction admixture,
c) maintaining said immunoreaction admixture for a time period sufficient for allowing anti-Corona antibodies against said Corona antigens in the body fluid sample to immunoreact with said Corona antigens to form an immunoreaction product,
d) separating the liquid phase from the solid phase
e) detecting the presence of any of said immunoreaction product in the solid or liquid phase or both.

Finally, the presence of any of said immunoreaction product is detected in the solid or liquid phase or both.

In embodiments, the maximal total duration of the method for detecting Corona-antibodies is less than one hour, i.e. less than 60 minutes, in an embodiment less than 30 minutes, in a further embodiment less than 20 minutes, in an embodiment between 15 and 30 minutes, in an embodiment between 15 to 20 minutes. The duration includes pipetting the sample and the reagents necessary to carry out the assay as well as incubation time, optional washing steps, the detection step and also the final output of the result.

In a sixth aspect, the present invention relates to a method of identifying if a patient has been exposed to Corona virus infection in the past, comprising
a) forming an immunoreaction mixture by admixing a body fluid sample of the patient with a Corona virus antigen of the first aspect of the present invention, a composition of the second aspect of the present invention, or a Corona virus antigen obtained by the method of the third aspect of the present invention
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or absence of any of said immunoreaction product, wherein the presence of an immunoreaction product indicates that the patient as been exposed to Corona virus infection in the past.

In embodiments, the patient was exposed to Corona virus infection prior to performance of the present method. In particular, the patient was exposed to Corona virus infection at least 5 days prior to performance of the present method. In particular, the patient was exposed to Corona virus infection at least 10 days prior to performance of the present method. In particular, the patient was exposed to Corona virus infection at least 14 days prior to performance of the present method.

In a seventh aspect, the present invention relates to a method of differential diagnosis between an immune response in a patient due to natural Corona virus infection and an immune response due to vaccination, wherein the vaccination is based on S-, E—, or M-protein derived antigens, comprising
a) forming an immunoreaction mixture by admixing a body fluid sample of the patient with a Corona virus antigen of the first aspect of the present invention, a composition comprising the Corona Antigen of the first of the present invention, or a Corona virus antigen obtained by the method of the third aspect of the present invention
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or absence of any of said immunoreaction product, wherein the presence of an immunoreaction product indicates that the immune response in the patient is due to a natural Corona virus infection, and wherein the absence of a immunoreaction product indicates that the immune response in the patient is due to vaccination with S-, E—, or M-protein derived antigens.

In embodiments, the method allows to differentiate between patients who were infected naturally with a Corona virus and patients who were vaccinated against Corona virus, wherein the patients vaccinated against Corona virus were vaccinated with a vaccine using an antigen derived from Corona Virus S—, E—, or M-protein.

In embodiments, the patient infected with a natural Coronas virus was infected with SARS-Cov-1 or SARS-Cov-2, in particular with SARS-Cov-2.

In embodiments, the natural corona virus comprises the nucleocapsid protein.

In an eighth aspect, the present invention relates to the use of a Corona antigen according to the first aspect of the present invention, the composition of the second aspect of the present invention, or the Corona antigen obtained by the method of the third aspect of the present invention, in a high throughput in vitro diagnostic test for the detection of anti-Corona virus antibodies. In particular embodiments, the Corona antigen according to the first aspect of the present invention, the composition of the second aspect of the present invention, or the Corona antigen obtained by the method of the third aspect of the present invention, are used in method of the fourth aspect of the present invention or of the fifth aspect of the present invention.

In a ninth aspect, the present invention relates to a reagent kit for the detection of anti-Corona virus antibodies, comprising a Corona antigen according to the first aspect of the present invention, the composition of the second aspect of the present invention, or the Corona antigen obtained by the method of the third aspect of the present invention.

In embodiments, the reagent kit comprises in separate containers or in separated compartments of a single container unit, a Corona antigen according to the first aspect of the present invention, the composition of the second aspect of the present invention, or the Corona antigen obtained by the method of the third aspect of the present invention. In particular embodiments, the comprised Corona antigen is that is covalently coupled to biotin.

In embodiments, the reagent kit further comprises in separate containers or in separated compartments of a single container unit, microparticles, in particular microparticles coated with avidin or streptavidin.

In further embodiments, the present invention relates to the following items:
1. A Corona antigen suitable for detecting antibodies against Corona virus in an isolated biological sample comprising a Corona nucleocapsid specific amino acid sequence according to SEQ ID NO: 1, or a variant thereof, wherein said polypeptide comprises no further Corona virus specific amino acid sequences.
2. The Corona antigen of item 1, wherein the Corona virus is CoV-1 or CoV-2 virus, in particular CoV-2 virus.
3. The Corona antigen of item 1 or 2, wherein said antigen further comprises at least one chaperone, in particular 2 chaperones.
4. The Corona antigen of item 3, wherein said chaperone is selected from the group consisting of SlyD, SlpA, FkpA, and Skp.
5. The Corona antigen of items 2-4, wherein the chaperone is fused to the Corona nucleocapsid specific amino acid sequence at the N— and/or— C-terminus of the nucleocapsid.
6. The Corona antigen of items 1 to 5, where the polypeptide comprises a Corona nucleocapsid specific amino acid sequence according to SEQ ID NO: 1 and two SlyD chaperones.
7. The Corona antigen of any of items 1 to 6, which is soluble and immunoreactive.
8. The Corona antigen of any of claims 1 to 7, wherein the SARS CoV-2 Corona nucleocapsid variant comprises an amino acid sequence according to SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15.
9. The Corona antigen of any of items 1 to 8 further comprising a tag, in particular a tag allowing to detect the antigen (in particular Ru, in particular negatively charged Ru), and/or a tag to bind the antigen directly or indirectly to a solid phase (in particular an effector group which is part of a bioaffine binding pair, in particular biotin).
10. A composition comprising the Corona antigen of any of items 1 to 9.
11. The composition of item 10 comprising additional Corona antigens, in particular comprising Corona antigens comprising amino acid sequences of the E-protein, the M-protein, and/or the S-Protein or parts thereof.
12. A method of producing a Corona antigen specific for Corona virus nucleocapsid, said method comprising the steps of
   a) culturing host cells, in particular E. coli cells, transformed with an expression vector comprising operably linked a recombinant DNA molecule encoding a polypeptide according to any of items 1 to 9, in particular a recombinant DNA molecule comprising a sequence according to SEQ ID NO: 3
   b) expression of said polypeptide and
   c) purification of said polypeptide.
13. A method for detecting antibodies specific for Corona virus in an isolated sample, wherein a Corona antigen according to any of items 1 to 9, the composition of item 10-11, or a Corona antigen obtained by a method according to item 12 is used as a capture reagent and/or as a binding partner for said anti-Corona virus antibodies.
14. A method for detecting antibodies specific for Corona virus in an isolated sample said method comprising
   a) forming an immunoreaction mixture by admixing a body fluid sample with a Corona virus antigen according to any of items 1 to 9, the composition of item 10-11, or a Corona virus antigen obtained by the method of item 12
   b) maintaining said immunoreaction admixture for a time period sufficient for allowing antibodies present in the body fluid sample against said Corona virus antigen to immunoreact with said Corona virus antigen to form an immunoreaction product; and
   c) detecting the presence and/or the concentration of any of said immunoreaction product
15. A method for detecting antibodies specific for Corona virus in an isolated sample according to item 14, wherein said immunoreaction is carried out in a double antigen sandwich format comprising
   a) adding to said sample a first Corona antigen which can be bound directly or indirectly to a solid phase and carries an effector group which is part of a bioaffine binding pair, and a second Corona antigen which carries a detectable label, wherein said first and second Corona antigens bind specifically to said anti-Corona antibodies
   b) forming an immunoreaction admixture comprising the first antigen, the sample antibody and the second antigen wherein a solid phase carrying the corresponding effector group of said bioaffine binding pair is added before, during or after forming the immunoreaction admixture,
   c) maintaining said immunoreaction admixture for a time period sufficient for allowing anti-Corona antibodies against said Corona antigens in the body fluid sample to immunoreact with said Corona antigens to form an immunoreaction product,
   d) separating the liquid phase from the solid phase
   e) detecting the presence of any of said immunoreaction product in the solid or liquid phase or both.
16. A method for detecting antibodies specific for Corona virus in an isolated sample according to any of items 13-15, wherein the detected antibody is an IgA, IgG or IgM antibody, in particular an IgG antibody.
17. A method of identifying if a patient has been exposed to Corona virus infection in the past, comprising
   a) forming an immunoreaction mixture by admixing a body fluid sample of the patient with a Corona virus antigen any of items 1 to 9, the composition of item 10-11, or a Corona virus antigen obtained by the method of item 12,
   b) maintaining said immunoreaction admixture for a time period sufficient for allowing antibodies present in the body fluid sample against said Corona virus antigen to immunoreact with said Corona virus antigen to form an immunoreaction product; and
   c) detecting the presence and/or absence of any of said immunoreaction product, wherein the presence of an immunoreaction product indicates that the patient has been exposed to Corona virus infection in the past.
18. A method of differential diagnosis between an immune response due to natural Corona virus infection and an immune response due to vaccination, wherein the vaccination is based on S—, E—, or M-protein derived antigens, comprising
   a) forming an immunoreaction mixture by admixing a body fluid sample of the patient with a Corona virus antigen of the first aspect of the present invention, a composition comprising the Corona Antigen of the first of the present invention, or a Corona virus antigen obtained by the method of the third aspect of the present invention
   b) maintaining said immunoreaction admixture for a time period sufficient for allowing antibodies present in the body fluid sample against said Corona virus antigen to immunoreact with said Corona virus antigen to form an immunoreaction product; and
   c) detecting the presence and/or absence of any of said immunoreaction product,
   wherein the presence of an immunoreaction product indicates that the immuneresponse in the patient is due to a natural Corona virus infection, and wherein the absence of a immunoreaction product indicates that the immuneresponse in the patient is due to vaccination with spike protein derived antigens.
19. Use of a Corona antigen according to any of items 1 to 9, the composition of item 10-11, or of a Corona antigen obtained by the method of item 12 in a high throughput in vitro diagnostic test for the detection of anti-Corona virus antibodies.
20. Use of a Corona antigen according to any of items 1 to 9, the composition of item 10-11, or of a Corona antigen obtained by the method of item 12 in the method of item 13 to 18.
21. A reagent kit for the detection of anti-Corona virus antibodies, comprising a Corona antigen according to any of items 1 to 9, the composition of item 10-11, or a Corona antigen obtained by the method of item 12.
22. A reagent kit according to item 18 comprising in separate containers or in separated compartments of a single container unit at least microparticles coated with avidin or streptavidin, and a Corona antigen according to any of items 1 to 9, the composition of items 10-11, or obtained by a method according to item 12 that is covalently coupled to biotin.
23. A reagent kit according to item 13, comprising in separate containers or in separated compartments of a single container unit at least microparticles coated with avidin or streptavidin, and a p-capture binding partner that is covalently coupled to biotin.

The following examples and figures are provided to aid the understanding of the present invention, the true scope of which is set forth in the appended claims. It is understood that modifications can be made in the procedures set forth without departing from the spirit of the invention.

### EXAMPLES

### Example 1: Cloning and Purification of Corona Nucleocapsid Antigens

Cloning of Expression Cassettes

On the basis of the pET24a expression plasmid of Novagen (Madison, Wis., USA), expression cassettes encoding fusion proteins were obtained essentially as described (Scholz, C. et al., J. Mol. Biol. (2005) 345, 1229-1241). The sequences of the nucleocapsid antigen from SARS Corona virus 2 (SARS CoV-2) were retrieved from the GenBank no. MN90847.3. A synthetic gene encoding the nucleocapsid antigen aa 1-419 (i.e., the full-length version of the nucleocapsid or N protein) with a glycine-rich linker region fused in frame to the N-terminus was purchased from Eurofins (Regensburg, Germany). Since the natural amino acid sequence of the Corona N protein does not contain any cysteine residues, no amino acid substitutions had to be made in order to prevent unwanted side-effects such as oxidation or intermolecular disulfide bridging. BamHI and XhoI restriction sites were at the 5′ and the 3′ ends of the N-coding region, respectively. A further synthetic gene encoding one or two EcSlyD units (residues 1-165 of SwissProt accession no. POA9K9) connected via a glycine-rich linker region and encompassing part of a further linker region at the C-terminus were likewise purchased from Eurofins. NdeI and BamHI restriction sites were at the 5′ and 3′ ends of this cassette, respectively. The genes and the restriction sites were designed to enable the in frame fusion of the chaperone part EcSlyD-EcSlyD and the N antigen part by simple ligation. In order to avoid inadvertent recombination processes and to increase the genetic stability of the expression cassette in the E. coli host, the nucleotide sequences encoding the EcSlyD units were degenerated as were the nucleotide sequences encoding the extended linker regions. i.e., different codon combinations were used to encode identical amino acid sequences.

The pET24a vector was digested with NdeI and XhoI and the cassette comprising tandem-SlyD fused in frame to Corona nucleocapsid (1-419) was inserted. Expression cassettes comprising E. coli SlpA (2-149, SwissProt ID POAEMO) E. coli Skp (21-161, SwissProt ID P0AEU7) or E. co/i FkpA (26-270, SwissProt ID P45523) were constructed accordingly, as well as expression cassettes comprising nucleocapsid fragments from SARS Corona virus 2. All recombinant fusion polypeptide variants contained a C-terminal hexahistidine tag to facilitate Ni-NTA-assisted purification and refolding. QuikChange (Stratagene, La Jolla, Calif., USA) and standard PCR techniques were used to generate point mutations, deletion, insertion and extension variants or restriction sites in the respective expression cassettes.

FIG. 3 shows a scheme of the Nucleocapsid antigen N 1-419 bearing two SlyD chaperone units fused in frame to its N-terminal end. To denote the E. coli origin of the SlyD fusion partner, the depicted fusion polypeptide has been named EcSlyD-EcSlyD-CoV-2 N (1-419).

The insert of the resulting plasmid was sequenced and found to encode the desired fusion protein. The complete amino acid sequences of the antigen variants CoV-2 N (1-419), EcSlyD-CoV-2 N (1-419) and EcSlyD-EcSlyD-CoV-2 N (1-419) are shown in SEQ ID NOs. 1, 2, and 3, respectively. The amino acid sequence of the linker L is shown is SEQ ID NO: 7.

Purification of recombinant proteins comprising nucleocapsid from SARS Corona Virus 2

All nucleocapsid antigen variants were purified by using virtually identical protocols. E. coli BLR (DE3) cells harboring the particular pET24a expression plasmid were grown at 37° C. in LB medium plus kanamycin (30 μg/ml) to an OD₆₀₀ of 1.5, and cytosolic overexpression was induced by adding 1 mM isopropyl-β-D-thiogalactoside. Three hours after induction, cells were harvested by centrifugation (20 min at 5000 g), frozen and stored at −20° C. For cell lysis, the frozen pellet was resuspended in chilled 50 mM sodium phosphate pH 8.0, 7.0 M GdmCl, 5 mM imidazole and the suspension was stirred for 2 h on ice to complete cell lysis. After centrifugation and filtration (0.45 μm/0.2 μm), the crude lysate was applied onto a Ni-NTA column equilibrated with the lysis buffer including 5.0 mM TCEP. The subsequent washing step was tailored for the respective target protein and ranged from 5 to 15 mM imidazole (in 50 mM sodium phosphate pH 8.0, 7.0 M GdmCl, 5.0 mM TCEP). At least 10-15 volumes of the washing buffer were applied. Then, the GdmCl solution was replaced by 50 mM potassium phosphate pH 8.0, 100 mM KCl, 10 mM imidazole, 5.0 mM TCEP to induce conformational refolding of the matrix-bound protein. In order to avoid reactivation of co-purifying proteases, a protease inhibitor cocktail (Complete© EDTA-free, Roche) was included in the refolding buffer. A total of 15-20 column volumes of refolding buffer were applied in an overnight reaction. Then, both TCEP and the Complete© EDTA-free inhibitor cocktail were removed by washing with 3-5 column volumes 50 mM potassium phosphate pH 8.0, 100 mM KCl, 10 mM imidazole. Subsequently, the imidazole concentration—still in 50 mM potassium phosphate pH 8.0, 100 mM KCl—was raised to 30-50 mM (depending on the respective target protein) in order to remove unspecifically bound protein contaminants. The native protein was then eluted by 250 mM imidazole in the same buffer. Protein-containing fractions were assessed for purity by Tricine-SDS-PAGE and pooled. Finally, the proteins were subjected to size-exclusion-chromatography (Superdex HiLoad, Amersham Pharmacia) and the protein-containing fractions were pooled and concentrated to 10-20 mg/ml in an Amicon cell (YM10).

After the coupled purification and refolding protocol, protein yields of roughly 10-15 mg could be obtained from 1 g of E. coli wet cells, depending on the respective target protein (unchaperoned N protein ˜ 10 mg/g; EcSlyD-N(1-419) ˜ 12 mg/g; EcSlyD-EcSlyD-N(1-419) ˜ 15 mg/ml).

### Example 2: Spectroscopic Measurements

Protein concentration measurements were performed with an Uvikon XL double-beam spectrophotometer. The molar extinction coefficients (ε₂₈₀) were determined by using the procedure described by Pace (1995), Protein Sci. 4, 2411-2423. The molar extinction coefficients (ε_{M280}) used for the distinct fusion polypeptides are specified in table 1.

TABLE 1
Protein parameters of the SARS Corona virus 2 nucleocapsid fusion
polypeptide variants generated and used in this study. All parameters
are referring to the respective protein monomers.

| | | molecular | | | |
|---|---|---|---|---|---|
| | length of | weight of | | | |
| | target | fusion | | | |
| fusion | protein (aa | polypeptide | | ε_{M280} | Abs_{0.1%} |
| protein | residues) | (Da) | pI | M⁻¹cm⁻¹ | (=1 mg/ml) |
| N variants | | | | | |
| SARS-CoV- | | | | | |
| 2 | | | | | |
| CoV-2 N | 2-419 | 46560 | 10.0 | 43890 | 0.943 |
| EcSlyD- | 1-419 | 66132 | 7.1 | 49850 | 0.754 |
| CoV-2 N | | | | | |
| EcSlyD- | 1-419 | 85442 | 5.8 | 55810 | 0.653 |
| EcSlyD- | | | | | |
| CoV-2 N | | | | | |

The unchaperoned SARS CoV-2 N was cloned in the full-length version (1-419), yet the N-terminal methionine is cleaved off co-translationally by the N-methionyl-aminopeptidase upon overproduction in E. coli. Therefore, the data for the mature (cleaved) SARS CoV-2 nucleocapsid version (2-419) are given in Table 1. The amino acid sequences of the Corona antigen variants are shown in SEQ ID NOs: 1, 2, and 3, respectively.

### Example 3: Coupling of Biotin Tag and Ruthenium Complex Label to the Nucleocapsid Antigen

The lysine F-amino groups of the fusion polypeptides were modified at protein concentrations of 10-30 mg/ml with N-hydroxy-succinimide activated biotin and ruthenium label molecules, respectively. The label/protein ratio varied from 1:1 to 10:1 (mol:mol), depending on the respective fusion protein. The reaction buffer was 150 mM potassium phosphate pH 8.0, 100 mM KCl, 0.5 mM EDTA. The reaction was carried out at room temperature for 15 min and stopped by adding buffered L-lysine to a final concentration of 10 mM. To avoid hydrolytic inactivation of the labels, the respective stock solutions were prepared in dried DMSO (seccosolv quality, Merck, Germany). DMSO concentrations up to 25% in the reaction buffer were well tolerated by all fusion proteins studied. After the coupling reaction, unreacted free label was removed by passing the crude protein conjugate over a gel filtration column (Superdex 200 HiLoad).

### Example 4: Immunological Reactivity (i.e., Antigenicity) of Different Nucleocapsid Antigen Variants in an Anti-SARS CoV-2 Immunoassay

The immunological reactivity (i.e. the antigenicity) of the polypeptide fusion variants of the Corona nucleocapsid antigen was assessed in automated Elecsys® cobas e 411 analyzers (Roche Diagnostics GmbH). Elecsys® is a registered trademark of the Roche group. Measurements were carried out in the double antigen sandwich format.

Signal detection in Elecsys® and cobas automated analyzers is based on electrochemiluminescence. The biotin-conjugate (i.e. the capture-antigen) is immobilized on the surface of a streptavidin coated magnetic bead whereas the detection-antigen bears a complexed Ruthenium cation (switching between the redox states 2+ and 3+) as the signaling moiety. In the presence of a specific immunoglobulin analyte, the luminescent ruthenium complex is bridged to the solid phase and emits light at 620 nm after excitation at a platinum electrode. The signal output is in arbitrary light units.

The recombinant Corona nucleocapsid antigens were assessed in a double antigen sandwich (DAGS) immunoassay format. To this end, recombinant Corona N antigen was used as a biotin and a ruthenium conjugate, respectively, to detect anti-Corona nucleocapsid antibodies in human sera.

The nucleocapsid protein N is one of the immunodominant antigens of Corona viruses, and soluble variants of N—as disclosed in this patent application—are invaluable tools for the detection of Corona infections. In all measurements, either EcSkp-EcSlyD-EcSlyD (EP2893021(B1)) or chemically polymerized and unlabeled EcSlyD-EcSlyD were implemented in large excess (5-30 μg/ml) in the reaction buffer as anti-interference substances to avoid immunological cross reactions via the chaperone fusion units.

In particular, three nucleocapsid variants from SARS Corona virus 2 were scrutinized in this study, namely full length N (1-419) without any fusion partner, full length N (1-419) fused to one SlyD chaperone and full length N (1-419) fused to two SlyD chaperone units. In order to detect both anti-SARS CoV-2N IgM and IgG molecules, EcSlyD-EcSlyD-N(1-419)-biotin and EcSlyD-EcSlyD-N-ruthenium were used in R1 (reagent buffer 1) and R2 (reagent buffer 2), respectively. The concentrations of the antigen conjugates in R1 and R2, respectively, were ˜100 ng/ml each (if not indicated otherwise). In analytical gel filtration experiments, we had found that EcSlyD-EcSlyD-N(1-419) forms soluble and regular oligomers, which display an epitope density which is high enough for the binding and detection of immunoglobulins of the M-type.

Furthermore, EcSlyD fusion polypeptides of putative immunodominant fragments of Corona antigens were assessed in Elecsys® measurements. Notably, fragments of the Spike protein (617-649, 338-516), of the E protein (8-65, 45-75), the M protein (1-32, 132-163, 100-222) and the N protein (151-178, 374-404) were examined for their antigenicity. All of these chaperone fusion proteins had been cloned, purified, biotinylated and ruthenylated, respectively, virtually as described for the N variants. The fragments had been chosen because there were hints in the literature that the corresponding sequences from SARS-CoV-1 were immunologically reactive. Indeed, for SARS-CoV-1, immunodominant epitopes have been described for the Corona Spike protein (He et al., J. Immunol. (2004); 173: 4050-4057), for the Corona M protein (J. Clin. Microbiol. (2005); 43(8): 3718-3726) and for the Corona N protein (J. Clin. Microbiol. (2004) 42 (2): 5309-5314).

Unfortunately, human Corona seroconversion panels—which are an indispensable tool for the development of improved in vitro diagnostic assays—have not yet been available commercially. In order to assess the antigenic properties of the different nucleocapsid variants in early phases of SARS CoV-2infection, we had to recur to remainder sera from clinics and hospitals.

In a first experiment, all of the Corona antigen candidates have been assessed for their immunological reactivity in the aforementioned DAGS format. To this end, the biotinylated and ruthenylated variant of the antigen candidate under study was incubated with the sample prior to addition of the streptavidin-coated beads. It is evident based on the data in FIG. 4A and FIG. 4B that the recombinant fusion polypeptides comprising fragments of the Corona proteins do not display any immunological reactivity: even at concentrations as high as 500 ng/ml, the Spike protein fragment 617-649 is not reactive at all with the five sera of the anti-Corona positive panel tested (see FIG. 4A). The detected signals are in the range of the system-inherent background which lies around 500 counts, ruling out that Spike (617-649) harbors immunodominant epitopes. The same holds true for another fragment from the Spike protein, namely 338-516, which encompasses the so-called receptor— binding domain and is supposed to be one of the most immunodominant regions within the Corona proteome. Also the recombinant-derived RBD Variant EcSlyD-Spike (338-516) does not show any reactivity, which is in strong contrast to previous reports on the antigenicity of this domain. The E protein variants (45-75) and (8-65)-both of them fused the the solubility-enhancing E. coli SlyD protein—do not show any reactivity either, as do the fragments 1-32, 132-163 and 100-222 of the Corona M protein. The results for the 100-222 region of the M protein are remarkable since this is the endodomain part of the M protein, i.e. there was a certain likelihood that this fragment is able to adopt a native-like conformation and thus would present conformational epitopes. Yet, there is no reacitivity at all for the M endodomain. There is no reactivity for the N fragments 151-178 and 374-404 either (FIG. 4B). In contrast, a weak but significant immunological reacitivity is revealed for the full-length nucleocapsid antigen (penultimate column), albeit at very high background signals. When a SlyD unit is fused N-terminally to the nucleocapsid, the solubility of the resulting fusion polypeptide is significantly enhanced and the background signals are lowered from ˜ 490000 counts to 120000 counts (FIG. 4B, last column).

As a result, the signal to noise ratio is significantly increased and the anti-Corona positive sera can be distinguished very well from the negative sera. Still, the background signal is very high, but can be mitigated by lowering the antigen concentration in the assay.

FIG. 4B shows that fusion of one SlyD unit to the SARS CoV-2nucleocapsid antigen conveys solubility to its target protein and improves its physicochemical properties, yielding an immunoreactive Corona antigen that is well-suited for the detection of anti-Corona antibodies.

In a next step, we explored whether fusion of another SlyD unit would further improve the physicochemical features of the nucleocapsid antigen.

FIG. 5 shows the Elecsys® assessment of the CoV-2 nucleocapsid antigen both in an unchaperoned form and fused to one SlyD unit and fused to two SlyD units. In order to ensure a fair comparison, identical molar concentrations of the respective variants were applied. Strikingly, by adding one SlyD chaperone unit, the background signal is significantly reduced and the signal-to-noise ratio, as a consequence, is improved. When a second SlyD chaperone unit is added to the Corona nucleocapsid antigen, the background signal ameliorates further and the s/n ratio is further increased. In brief, the solubility of the Corona N protein strongly benefits from the fusion of chaperones such as SlyD. And it is evident from the comparison of FIG. 5 that even the signal recovery is markedly improved when two SlyD units are added to N instead of only one. Long-term stability is a critical issue and a prerequisite for any antigen that is used in an immunoassay. The signal recovery and, actually, the signal-to-noise recovery should not be severely affected when the antigen is incubated under thermal stress conditions such as, e.g. 35° C. Table 5 also shows that fusion of two SlyD chaperone units to the Corona N antigen improves the overall signal recovery and renders N usable in an Elecsys® DAGS format for the reliable detection of anti-Corona antibodies. After an over-night-incubation at 35° C., the s/n recovery is much higher with the EcSlyD-EcSlyD-CoV-2-N conjugates than it is with the unchaperoned CoV-2 N conjugate. We found that the same holds true for SlpA (SlyD-like protein A)-N fusion proteins. E. coli SlpA is a close relative of E. coli SlyD and has very advantageous properties with respect to thermal stability (see Example 7 below).

Further optimizations with respect to anti-interference additives, buffer composition and antigen concentration in R1 (=reagent 1; biotin conjugates) and R2 (=reagent 2; ruthenium conjugates) as well as an adsorptive pretreatment of the ruthenium conjugate with beads (FIG. 6) finally paved the way for an Elecsys®-compatible nucleocapsid antigen with excellent background values (i.e., very low signals with negative sera) and outstanding signal-to-noise ratios (s/n) which facilitate a good discrimination between anti-Corona positive and negative sera.

Taken together, we conclude that fragments of the Corona proteins that have been touted as immunodominant epitopes, be it linear (such as Spike 617-649) or conformational (such as the receptor binding domain RBD that is contained within the spike protein), do not show significant antigenicity in our hands. When assessed in an Elecsys® automated analyzer, we did not find any antigenicity with the promising Corona protein fragments, but only with the full-length nucleocapsid antigen from CoV-2. Yet, in its natural form, the N protein was not usable in the Elecsys® assay due to excessive background signals. Fusion of two SlyD chaperone units to the N antigen cured this drawback and rendered the N antigen suitable for high-throughput applications on the Elecsys® platform.

### Example 5: Sensitivity and Specificity of the Anti-SARS CoV-2 Immunoassay as Described Above

Initially, 129 patients identified by PCR analysis to be infected with SARS CoV-2 were further examined by means of our prototype antibody immunoassay based on the nucleocapside antigen. At different time intervals after the positive PCR test, serum samples were taken and analyzed via the above-described antibody assay to elucidate whether there were any anti-CoV-2 antibodies present in the sample. The results were grouped into 3 categories: below 7 days after positive PCR, between 7 and 13 days and 14 days and longer after initial PCR result.

Six days after a positive PCR test, 74% of patients could be identified as being anti-SARS CoV-2 positive. Between 7 and 13 days post PCR positivity, already 95% of the patients were identified as SARS CoV-2 positive. 14 days after PCR positivity, our assay detects 100% of all patients as being positive. Results are also illustrated in FIG. 7A).

In additional experiments, a total of 204 samples from 69 symptomatic patients with a PCR confirmed SARS CoV-2infection were tested with the Elecsys Anti-SARS CoV-2 assay as described above. One or more consecutive specimens from these patients were collected after PCR confirmation at various time points. Results are also illustrated in FIG. 7B).

In a third experiment, additional 292 samples from 61 symptomatic patients with a PCR confirmed SARS CoV-2 infection were tested with the Elecsys Anti-SARS CoV-2 assay as described above. One or more consecutive specimens from these patients were collected after PCR confirmation at various time points. Results are also illustrated in FIG. 7C). One sample was non-reactive after 14 days, but turned reactive after 16 days. Thus, after 16 days also for this data set, the sensitivity is 100%.

For specificity testing, initially 1591 diagnostic routine serum and plasma samples taken prior to December 2019 (“pre-pandemic samples”) were analyzed by means of above-described antibody assay. Due to the donation date all samples were classified as SARS CoV-2 antibody negative. Out of the 1591 samples only 2 were identified as anti-SARS CoV-2 reactive. Thus, the above described antibody assay has a specificity of 99.87%.

In additional experiments, further patient samples were analysed. A first set of samples from 5272 patients analysed which include the above named initial 1591 samples. The following samples were enclosed
3420 samples from patients in diagnostic routine
1772 samples from blood donors
40 samples from patients with diagnosed common cold panel, and
40 potentially cross reactive samples from patients with past infection with Coronavirus HKU1, NL63, 229E or OC43, confirmed via PCR.

All samples were obtained before December 2019 and were tested with the Elecsys Anti-SARS CoV-2assay as described above. 10 false positive samples were detected.

The resulting overall specificity in the first sample set was 99.81%. The 95% lower confidence limit was 99.65%. Results are illustrated in FIG. 8A.

In a second set, additional 5261 samples from patients were analysed. The following samples were enclosed in the specificity study
2376 samples from patients in diagnostic routine
2885 samples from blood donors

Furthermore, samples from 4696 Dialysis patients were analysed.

The resulting overall specificity in the second sample set was 99.79%. The 95% lower confidence limit was 99.63%. Results are illustrated in FIG. 8B.

The combined results of the first and second set of sample measurements (in total 10453) are illustrated in FIG. 8C.

### Example 6: Capillary Blood as Suitable Sample Type for the Anti-SARS CoV-2 Immunoassay as Described Above

To analyze whether capillary blood would be suitable to be used as sample type in the above described anti-SARS CoV-2 immunoassay, capillary blood samples were compared to serum samples prepared from venous blood. Also the effects of three different anti-coagulants were analysed (Li Heparin Plasma, K2-EDTA Plasma, CAT Serum). For Li Heparin Plasma, K2-EDTA Plasma, 10 samples were tested, 5 of which were positive and 5 were negative. For CAT Serum 7 samples were tested, 5 of which were positive and 2 were negative. Results are summarized in Tables 2, 3, and 4 below, and in FIGS. 9A, B, and C, respectively.

TABLE 2
Correlation between venous serum sample
and capillary Li Heparin plasma sample

| | | | Lithium- | | |
|---|---|---|---|---|---|
| | | | Heparin- | | |
| | | | Plasma | COI | COI |
| | | Serum | (capillary) | recovery | recovery |
| Sample | target | COI | COI | [abs] | [%] |
| Sample1 | neg | 0.049 | 0.046 | 0.003 | — |
| Sample2 | neg | 0.047 | 0.045 | 0.002 | — |
| Sample3 | pos | 69.8 | 67.1 | — | 96% |
| Sample4 | pos | 87.5 | 88.1 | — | 101% |
| Sample5 | neg | 0.052 | 0.050 | 0.002 | — |
| Sample6 | pos | 16.0 | 14.9 | — | 93% |
| Sample7 | neg | 0.048 | 0.068 | 0.020 | — |
| Sample8 | neg | 0.049 | 0.046 | 0.003 | — |
| Sample9 | pos | 10.4 | 9.37 | — | 90% |
| Sample10 | pos | 10.1 | 9.30 | — | 92% |
| | | | MIN | 0.002 | 90% |
| | | | MAX | 0.020 | 101% |
| | | | MEAN | 0.011 | 95% |

TABLE 3
Correlation between venous serum sample
and capillary K2-EDTA plasma sample

| | | | K2-EDTA- | | |
|---|---|---|---|---|---|
| | | | Plasma | COI | COI |
| | | Serum | (capillary) | recovery | recovery |
| Sample | target | COI | COI | [abs] | [%] |
| Sample1 | neg | 0.050 | 0.050 | 0.0003 | — |
| Sample2 | pos | 1.21 | 1.38 | — | 114% |
| Sample3 | neg | 0.957 | 1.12 | 0.165 | — |
| Sample4 | pos | 1.20 | 1.23 | — | 102% |
| Sample5 | neg | 0.811 | 0.874 | 0.063 | — |
| Sample6 | neg | 0.048 | 0.048 | 0.0002 | 100% |
| Sample7 | pos | 5.03 | 3.51 | — | 70% |
| Sample8 | pos | 4.08 | 4.13 | — | 101% |
| Sample9 | pos | 6.15 | 6.28 | — | 102% |
| Sample10 | pos | 5.80 | 5.08 | — | 88% |
| | | | MIN | 0.000 | 70% |
| | | | MAX | 0.165 | 114% |
| | | | MEAN | 0.083 | 92% |

TABLE 4
Correlation between venous serum sample
and capillary CAT Serum sample

| | | | Serum | COI | COI |
|---|---|---|---|---|---|
| | | Serum | (capillary) | recovery | recovery |
| Sample | target | COI | COI | [abs] | [%] |
| Sample1 | neg | 0.066 | 0.069 | 0.003 | — |
| Sample2 | pos | 1.34 | 1.42 | — | 106% |
| Sample3 | pos | 1.11 | 1.26 | — | 113% |
| Sample4 | pos | 1.37 | 1.10 | — | 81% |
| Sample5 | neg | 0.071 | 0.068 | 0.003 | — |
| Sample6 | pos | 10.6 | 10.0 | — | 95% |
| Sample7 | pos | 4.47 | 4.35 | — | 97% |
| | | | MIN | 0.003 | 81% |
| | | | MAX | 0.003 | 113% |
| | | | MEAN | 0.003 | 97% |

To address sample volume variations among capillary blood, venous whole blood (collected w/o clot activator or anti-coagulant) was transferred in different volumes into a capillary collection tube with anticoagulant (300 μl, 400 μl, 600 μl, 800 μl =Reference), centrifuged and tested on cobas e analyzers with Elecsys Anti-SARS-CoV-2. One negative sample and one spiked positive sample was tested. Results are illustrated in Table 5 below.

TABLE 5
Effects of sample volume variations

| | | | | COI | | COI | | COI |
|---|---|---|---|---|---|---|---|---|
| | | 800 μl | 300 μl | recovery | 400 μl | recovery | 600 μl | recovery |
| Sample | target | COI | COI | [abs/%] | COI | [abs/%] | COI | [abs/%] |
| Sample1 | neg | 0.050 | 0.048 | 0.001 | 0.049 | 0.001 | 0.050 | 0.000 |
| Sample2 | pos | 5.66 | 5.54 | 98% | 5.68 | 100% | 5.72 | 101% |

| | COI | COI |
|---|---|---|
| | recovery | recovery |
| | [abs] | [%] |
| MIN | 0.000 | 98% |
| MAX | 0.001 | 101% |
| MEAN | 0.001 | 99% |

### Example 7: Nucleocapsid Antigen Fused to Alternative Chaperone

Using the same method as described above in Examples 1 to 3, the nucleocapsid sequence from SARS Corona virus 2 (SARS CoV-2) was also fused to an alternative chaperone, namely SlpA. The resulting fusion polypeptide was coupled to either biotin tag or ruthenium complex label. The immune reactivity was tested as described in Example 4 above and compared to the reactivity of the aforementioned SlyD-antigen construct. Results are shown in FIG. 10.

### Example 8: Differential Diagnosis of SARS CoV-2 vs. Common Cold Coronaviruses 229E, OC43, NL63 and HKU1

In addition to the nucleocapsid antigen from SARS-CoV-2, it should be worthwhile to have at hand the nucleocapsid homologues from the other six well-known human pathogenic Coronaviruses, namely 229E, OC43, SARS-CoV-1, NL63, HKU1 and MERS (listed in the order of their appearance in the scientific literature). The so-called common cold coronaviruses 229E, OC43, NL63 and HKU1 are still circulating in the human population worldwide and are—especially in the winter season—the etiological agent of cold-like diseases (Human coronavirus circulation in the United States 2014-2017, J. Clin. Virol. 101 (2018), 52-56). With the respective antigens at hand, it should be possible to facilitate both anti-interference approaches of the anti-SARS CoV-2immunoassay and a differential diagnosis of questionable sera under study. When, for instance, a serum that is false positive with the anti-SARS CoV-2assay is reactive with the rec. EcSlyD-EcSlyD-N constructs from 229E and NL63 (both alpha-coronaviruses), it is mandatory to rule out that antibodies raised upon a relatively harmless alpha-coronaviral infection did crossreact with the rec. EcSlyD-EcSlyD-CoV-2-N specifier that is used in the anti-SARS CoV-2antibody test, thus erroneously indicating a SARS CoV-2infection. By means of either specific blocking experiments (i.e., the addition of unlabeled common cold corona N antigens to the sample under scrutiny) or differential diagnosis of anti-CoV-2 reactive samples with labeled N variants from the common cold Coronaviruses, it should be possible to confirm a true positive result or to rule it out, respectively.

Therefore, we cloned, expressed and purified (in E. coli B121) the rec. EcSlyD-EcSlyD-fusion protein versions of the N-antigens from 229E, OC43, SARS-CoV-1, NL63, HKU1 and MERS as described for rec. EcSlyD-EcSlyD-N from SARS-CoV-2. All N variants—except OC43 and HKU1—could be obtained in high yields from E. coli and turned out to be soluble and stable in our hands. Protein data and yields are summarized in Table 6.

TABLE 6
protein features of the EcSlyD-EcSlyD-N antigen variants that were cloned,
expressed and examined in this study. The nucleocapsid proteins of the seven
known human pathogenic Coronaviruses were constructed as EcSlyD-EcSlyD fusion
proteins and essentially purified as described in the examples section.

| | | | | Length | | | | | |
|---|---|---|---|---|---|---|---|---|---|
| | | | | SS-N | | | | | |
| human | | Year or | Length | Fusion | | | SS-N | | SS-N |
| pathogenic | | first | N-antigen | antigen | SS-N | SS-N | A₂₈₀ | SS-N | MW |
| Corona Virus | | description | (AS) | (AS) | (mg) | (M⁻¹cm⁻¹) | (0.1%) | pI | (Da) |
| 229E | alpha | 1965 | 1-389 | 772 | 50 | 49 850 | 0.599 | 5.70 | 83 251 |
| OC43 | beta | 1967 | 1-448 | 831 | 14 | 61 770 | 0.694 | 5.52 | 89 068 |
| SARS-CoV-1 | beta | 2002 | 1-422 | 805 | 36 | 55 810 | 0.650 | 5.85 | 85 841 |
| NL63 | alpha | 2004 | 1-377 | 760 | 51 | 49 850 | 0.608 | 5.58 | 82 037 |
| HKU1 | beta | 2005 | 1-441 | 824 | 12 | 63 385 | 0.713 | 5.64 | 88 918 |
| MERS | beta | 2012 | 1-413 | 796 | 42 | 59 820 | 0.705 | 5.80 | 84 879 |
| SARS-CoV-2 | beta | 2019 | 1-419 | 802 | 50 | 55 810 | 0.653 | 5.83 | 85 442 |

Furthermore, we cloned, expressed and purified from an E. coli BL21 overproducer the so-called N-terminal domains (NTD) of the N proteins from SARS-CoV-2, 229E, OC43, NL63 and HKU1, according to a purification protocol that was essentially identical to the one described for the full-length N version rec. EcSlyD-EcSlyD-CoV-2-N. In contrast to the full-length N protein, the N-terminal domain does not form dimers and tetramers, but is strictly monomeric. Therefore, the NTD is particularly suited for the detection of immunoglobulins of the G-type. Immunoglobulins of the M-type are, however, not recognized by the strictly monomeric NTD when the antigen is used as a capture and a detection molecule in a double antigen sandwich (DAGS) format. Physiologically, the NTD binds to and accommodates the polyanionic single-stranded viral RNA polymer within the Corona virion. We could show that the solubility of the NTD is dramatically improved when compared to the full-length N protein and that its thermally induced unfolding is—in marked contrast to the full-length N antigen—fully reversible. Melting curves monitored by near-UV CD spectroscopy showed a very favourable folding behaviour of rec. EcSlyD-N_NTD, since the near-UV CD signal is fully restored after a thermal 20° C.−80° C.−20° C. unfolding/refolding cycle (data not shown), indicating a high solubility of both the unfolded state and of potential folding intermediates. Reversibility of thermally induced unfolding is a very fortunate and welcome feature of a protein, and the lack of any aggregation tendency characterizes the NTD as an excellent antigen for use in an immunoassay. Protein data and yields of the various NTD constructs are depicted in Table 7.

TABLE 7
protein features of the EcSlyD-N_NTD antigen variants that were cloned, expressed and purified

| | | | | Length of | | | | | |
|---|---|---|---|---|---|---|---|---|---|
| | | | | S-NTD | | | | | |
| human | | Year of | | Fusion | | S-NTD | | | S-NTD |
| pathogenic | | first | Length | antigen | S-NTD | εM₂₈₀ | S-NTD | S-NTD | MW |
| Corona Virus | | description | of NTD | (AS) | (mg) | (M⁻¹cm⁻¹) | A₂₈₀ | pI | (Da) |
| 229E | alpha | 1965 | 19-144 | 322 | 113 | 31 400 | 0.894 | 5.81 | 35 130 |
| OC43 | beta | 1967 | 59-189 | 327 | 5 | 44 350 | 1.257 | 4.92 | 35 295 |
| NL63 | alpha | 2004 | 16-142 | 323 | 121 | 29 910 | 0.849 | 5.71 | 35 210 |
| HKU1 | beta | 2005 | 58-188 | 327 | 104 | 42 860 | 1.213 | 5.23 | 35 347 |
| SARS-CoV-2 | beta | 2019 | 46-176 | 327 | 63 | 32 890 | 0.944 | 5.37 | 34 831 |

All rec. EcSlyD-N_NTD variants were biotinylated and ruthenylated as described. The respective pairs of biotin- and ruthenium conjugates showed excellent background signal in the Elecsys® assessment and were well-suited to discriminate between positive and negative sera. In FIGS. 11A & 11B, the reactivities of the NTDs from SARS-CoV-2, OC43, NL63, 229E and HKU1 with human sera are depicted.

The sera had been partially pre-characterized by means of the recomLine lateral flow assay SARS CoV-2 IgG[Avidity]RUO (article no. 7374, Mikrogen GmbH, Neuried, Germany), because reliable figures on the actual seroprevalence of antibodies to the common cold coronaviruses have not been available. To put it simply, we wanted to make sure that among the sera under study there was at least one which was negative for each of the four common cold coronaviruses. FIGS. 11A & 11B illustrate that we do not observe any immunoreactivity against the novel pathogen SARS CoV-2 in the pre-pandemic common cold corona panel dating from 2019 (FIGS. 11A & 111B, column 1). All of the CCC sera are anti-SARS CoV-2 negative, yielding electrochemiluminescence signals near the system-inherent background (450-600 counts). As for the anti-SARS CoV-2 positive panel (dating from 2020), the signals of the SARS CoV-2 NTD are markedly reduced with respect to the full-length version of SARS CoV-2-N. This was expected, since the NTD (46-176) is devoid of the complete C-terminal part of the molecule (177-419) and therefore lacks many of the natural epitopes. Besides, due to the strictly monomeric character of the NTD, many of the anti-Corona-antibodies in the polyclonal patient sera which may be targeted towards conformationally folded dimeric and higher oligomeric versions of N, are no more able to recognize and bind to their target molecule. Yet, the rather poor signal level that we observe with the SARS CoV-2-NTD seems still to be sufficient to reliably distinguish between positive and negative sera (FIGS. 11A & 11B, column 1). This finding also holds true for the common cold coronavirus (CCC) NTDs from OC43 and HKU1 (FIGS. 11A & 11B, columns 2 & 5). As can be deduced from column 2, the prevalence of antibodies towards OC43 seems to be rather moderate. For this beta-coronavirus, we find—in the Elecsys® assessment—many sera with background signals near the system-inherent background. This points to an excellent solubility of the OC43 antigen in general and the OC43 antigen-ruthenium conjugate in particular. Remarkably, we also find sera with a high signal level and fairly good signal dynamics, enabling an excellent discrimination between positive and negative sera. As for NL63 and 229E, we could not find—in a first trial—true negative sera with signals in the range of the system-inherent background. All sera tested seem to be anti-CoV positive for both NL63 and 229E (FIGS. 11A & 11B, columns 3 & 4), and the signal dynamics are very high. The true positivity of the sera is corroborated by a reference measurement, in which the human sera were substituted by buffer and a universal diluent on the sample position, respectively. In this experimental setting, the NTD of both NL63 and 229E turned out to exhibit outstandingly low background signals in the range of 400-650 counts (FIG. 111B, lower three “buffer” rows). This finding is important in two regards: firstly, it rules out specific or unspecific association reactions between the biotinylated and the ruthenylated NL63 and 229E NTD molecules in the assay, which would lead to a dramatic increase in signal. Secondly, it confirms that the NTD-ruthenium conjugate of both NL63 and 229E are highly soluble and do not tend to bind to the surface of the streptavidin-coated beads in the Elecsys® assay. Taken together, the data suggest that the high signals of NL63 and 229E measured with human sera are true and valid results, underlining a very high prevalence of antibodies against the coronaviruses NL63 and 229E—much higher than for OC43. The picture is completed by the data for HKU1 (FIGS. 11A & 11B, column 5). The prevalence for this corona strain seems to be rather high, too, but in contrast to NL63 and 229E we do find true negative sera for HKU1, with signals near the system-inherent background. According to our very preliminary data and on the basis of the few sera tested, it is tempting to deduce a tentative prevalence ranking for the common cold coronaviruses OC43<HKU1<NL63, 229E in the analyzed panel.

Our result may be due to contingency because of the low number of sera tested, but it seems that the alpha-coronaviruses 229E and NL63 have been circulating in the analyzed cohort especially effectively in the precedent winter seasons, whereas OC43 infections seem to have been rather scarce.

By and large, the expression, purification and modification (i.e., biotinylation and ruthenylation) of the N-terminal domains of the N-antigens from the corona viruses SARS-CoV-2, OC43, NL63, 229E and HKU1 allowed us to establish a simple serological discrimination between the related common cold coronaviruses. Given that SARS CoV-2 is relentlessly spreading worldwide in an unprecedented pandemic, our approach may be an attractive option of a simple differential diagnosis that enables discrimination of a potentially life-threatening SARS CoV-2infection from a harmless cold induced by one of the four well-known common cold viruses OC43. NL63, 229E and HKU1.

### Example 8: Mutations of Wild Type SARS CoV-2 Nucleocapsid Antigen

Due to the increasing number of emerging SARS CoV-2 mutation variants, we generated four mutation variants containing 3, 8, 12, or 15 single point mutations (see FIG. 12) and expressed each of them fused via the linker of SEQ ID NO: 7 to two EcSlyD units as described above.

3 MUT: SEQ ID NO: 8
EcSlyD-EcSlyD- SARS CoV-2-N 3 MUT: SEQ ID NO: 9
8 MUT: SEQ ID NO: 10
EcSlyD-EcSlyD- SARS CoV-2-N 8 MUT: SEQ ID NO: 11
12 MUT: SEQ ID NO: 12
EcSlyD-EcSlyD- SARS CoV-2-N 12 MUT: SEQ ID NO: 13
15 MUT: SEQ ID NO: 14
EcSlyD-EcSlyD- SARS CoV-2-N 15 MUT: SEQ ID NO: 15

The introduced single point mutations correspond to the naturally occurring mutations of the SARS CoV-2 mutations currently circulating in the populations.

The most common circulating mutations are:

B.1.1.7 (UK): D3L, S235F

B.1.525 (UK/Nigeria): D3A, A12G, T205I

COH.20G/677H (Ohio): P67S, P199L, D377Y

B.1.351 (South Africa): T205I

P.1 (B.1.1.28.1): P80R, R203K

P.2 (Brazil): A119S

P.3 (Philippines): R203K, G204R

N.9 (Brazil): I292T

EPI_ISL_1360318 (India): R203M

In FIG. 12 the single amino acid exchanges which were included in the four mutation variants are indicated with patterned bars and specified amino acid exchange Further SARS CoV-2 nucleocapsid single-point mutations which have been found less often in the population than the ones named above, were also introduced and are indicated in FIG. 12 in black bars. These were chosen according to the CoV-GLU databased published at: http://cov-glue.cvr.gla.ac.uk/#/home (Update of Feb. 24, 2021, 17:11:05 GMT) as being amongst the most frequent mutations found in infected individuals worldwide.

Two variants containing either three (3 MUT), or eight (8 MUT) single point mutations were assessed for the impact of the selected mutations within the nucleocapsid protein on the performance of the detection. Therefore, we used the labeled form of the nucleocapsid variants in a double antigen sandwich (DAGS) immunoassay format as described above.

Sera from 50 individuals were tested in parallel with either the wild type EcSlyD-EcSlyD-nucleocapsid fusion protein or variants of the protein containing either three (3 MUT) eight (8 MUT) single point mutations. The mean COI recovery of the variants was calculated and compared to the wild type reactivity (see Table 8 and FIG. 13).

TABLE 8
Recovery of WT vs 3 MUT and 8 MUT

| n = 50 | WT | 3 MUT | 8 MUT |
|---|---|---|---|
| MIN | 100% | 88% | 71% |
| MAX | 100% | 108% | 123% |
| MEAN | 100% | 95% | 85% |
| SD | 0% | 4% | 12% |
| CV | 0% | 4% | 14% |

Introducing three single point mutations to the nucleocapsid protein sequence results in a very slightly reduced reactivity (5%) over all samples tested. Since these point mutations correspond to the amino acid substitutions found in the B.1.1.7 (D3L, S235F) and B.1.351 (T205I) variant of SARS-CoV-2, we conclude that the Elecsys anti-SARS-CoV-2 assay delivers valid results when applied with antisera from individuals infected by one of the widespread UK or South Africa variants. Even the exchange of as many as eight amino acids within the protein sequence still leads to a mean COI recovery of 85% and a higher variation of signals compared to the wild type sequence. Importantly, the closer the signal is to the Cutoff (COI=1.0), the smaller is the reactivity difference between the variants and the wild type nucleocapsid sequence, assuring that the classification of the sample as reactive or non-reactive is not impacted by one of the variants. In brief, despite the substitution of three (D3L, T205I, S235F) and eight (P67S, D103Y, S194L, G204R, A220V, M2341, H300Y, A376T) amino acid residues, respectively, within the nucleocapsid antigen, we observe almost wildtype reactivity in our N-based Elecsys® antibody assay. We conclude from these observations that positive antisera from individuals infected with one of the hitherto known SARS CoV-2 variants will be detected as positive at any rate.

Furthermore, we found that the 3 MUT, 8 MUT and 15 MUT variants adopt a native conformation (i.e., they are native-like folded) since their elution behavior in an analytical gel filtration (via a superdex 200 column) equals the elution behavior of the wild-type nucleocapsid antigen. In case of partial or global unfolding due to the introduced mutations, one would expect an apparent enlargement of the molecule. Our observation is that the N variants bearing 3, 8 and 15 mutations, respectively, maintain their global fold and show an elution behavior virtually identical to the wild-type N protein.

1. A Corona antigen suitable for detecting antibodies against Corona virus in an isolated biological sample comprising a Corona nucleocapsid specific amino acid sequence according to SEQ ID NO: 1, or a variant thereof, wherein said Corona antigen further comprises at least one chaperone, and wherein said antigen comprises no further Corona virus specific amino acid sequences.

2. The Corona antigen of claim 1, wherein the Corona virus is SARS-CoV-2 virus.

3. The Corona antigen of claim 1, wherein said chaperone is selected from the group consisting of SlyD, SlpA, FkpA and Skp.

4. The Corona antigen of claim 1, wherein the SARS CoV-2 Corona nucleocapsid variant comprises an amino acid sequence according to SEQ ID NO: 8, SEQ ID NO: 10, SEQ ID NO: 12, or SEQ ID NO: 14.

5. The Corona antigen of claim 1, wherein the Corona antigen comprises an amino acid sequence according to SEQ ID NO: 3, SEQ ID NO: 9, SEQ ID NO: 11, SEQ ID NO: 13, or SEQ ID NO: 15.

6. A composition comprising at least one Corona antigen of claim 1.

7. A method of producing a Corona antigen specific for Corona virus nucleocapsid, said method comprising the steps of
a) culturing host cells transformed with an expression vector comprising an operably linked recombinant DNA molecule encoding a polypeptide according to claim 1,
b) expressing said polypeptide and
c) purifying said polypeptide.

8. A method for detecting antibodies specific for Corona virus in an isolated sample, wherein a Corona antigen according to claim 1, the composition of claim 6, or a Corona antigen obtained by a method according to claim 7, is used as a capture reagent and/or as a binding partner for said anti-Corona virus antibodies.

9. A method for detecting antibodies specific for Corona virus in an isolated sample said method comprising
a) forming an immunoreaction mixture by admixing a body fluid sample with a Corona virus antigen according to claim 1, the composition of claim 6, or a Corona antigen obtained by a method according to claim 7,
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or the concentration of any of said immunoreaction product

10. A method of identifying if a patient has been exposed to Corona virus infection in the past, comprising
a) forming an immunoreaction mixture by admixing a body fluid sample of the patient with a Corona virus antigen of claim 1, the composition of claim 6, or a Corona antigen obtained by a method according to claim 7,
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or absence of any of said immunoreaction product, wherein the presence of an immunoreaction product indicates that the patient has been exposed to Corona virus infection in the past.

11. A method of differential diagnosis between an immune response due to natural Corona virus infection and an immune response due to vaccination, wherein the vaccination is based on S—, E—, or M-protein derived antigens, comprising
a) forming an immunoreaction mixture by admixing a body fluid sample with a Corona virus antigen according to claim 1, a composition of claim 6, or a Corona antigen obtained by a method according to claim 7,
b) maintaining said immunoreaction admixture for a time period sufficient for allowing antibodies present in the body fluid sample against said Corona virus antigen to immunoreact with said Corona virus antigen to form an immunoreaction product; and
c) detecting the presence and/or absence of any of said immunoreaction product,
wherein the presence of an immunoreaction product indicates that the immune_response in the patient is due to a natural Corona virus infection, and wherein the absence of a immunoreaction product indicates that the immune response in the patient is due to vaccination with spike protein derived antigens.

12. Use of a Corona antigen according to claim 1, the composition of claim 6, or a Corona antigen obtained by a method according to claim 7, in a high throughput in vitro diagnostic test for the detection of anti-Corona virus antibodies.

13. (canceled)

14. A reagent kit for the detection of anti-Corona virus antibodies, comprising a Corona antigen according to claim 1, the composition of claim 6, or a Corona antigen obtained by a method according to claim 7.
